(19) United States
(12) Patent Application Publication
(10) Pub. No.: US 2002/0106635 A1
(43) Pub. Date: Aug. 8, 2002

(54) CYTOKINE RESISTANT CYTOMEGALOVIRUS PROMOTER MUTANTS AND RELATED PRODUCTS AND METHODS

(76) Inventor: BRUCE FREIMARK, RESIDENCE (XP)

Correspondence Address: LYON & LYON LLP/ VALENTIS INC., 633 WEST FIFTH STREET, SUITE 4700, LOS ANGELES, CA, 90071-2066, US

(21) Appl. No.: 09/321,261

(22) Filed: May 26, 1999

Related U.S. Application Data
(60) Provisional application No. 60/087,044, filed on May 27, 1998.

(51) Int. Cl. C12Q 1/70

(52) U.S. Cl. 435/5

(57) ABSTRACT

The present invention relates generally to gene expression and regulation, and more particularly to cytokine resistant cytomegalovirus promoter mutants useful in gene therapy.

### INTRODUCTION

The present invention relates generally to gene expression and regulation, and more particularly to cytokine resistant cytomegalovirus promoter mutants useful in gene therapy.

### BACKGROUND OF THE INVENTION

The following discussion of the background of the invention is merely provided to aid the reader in understanding the invention and is not admitted to describe or constitute prior art to the present invention.

The use of strong constitutive viral promoters, such as the CMV immediate early (i.e.,) promoter, have been widely used in mammalian gene expression vectors for gene therapy. A major limitation of current plasmids used for in vivo transfections is short-term low level transgene expression. Several studies have demonstrated that the in vivo administration of DNA from bacterial and viral origin, which is commonly found in gene therapy expression vectors, stimulates the immune response to produce a variety of cytokines including IFNγ, TFNα and IL-12, Pisetsky, D. S., et al., Ann. N. Y. Acad. Sci. 772:152, 1995; Klinman, D., G. et al., J. Immunol. 158:3635, 1997; Raz, E., et al., Proc. Natl. Acad. Sci. U. S. A. 93:5141, 1996; Ghazizadeh, S., et al., J. Virology 71:9163, 1997.

The presence of IFNγ and TFNα have been shown to suppress transgene expression in cells transfected in vitro Harms, J. S. and G. A. Splitter. Hum. Gene Ther. 6:1291, 1995, and in vivo Qin, L., et al., Human Gene Therapy 8:2019, 1998, with CMV- or SV40-based promoter plasmids.

The human CMV enhancer/promoter is rich in cis-acting elements which are either repetitive or non-repetitive DNA sequence motifs. The enhancer has repeat elements of 17, 18, and 21 bps that contain NFκB/rel, CREB/ATF and both SP-1 and YY1 binding sites respectively. Non-repeated elements found in the enhancer are AP-1, serum response (SRE) and ETS elements. Several transcription proteins which bind the human CMV promoter/enhancer repress promoter activity which include the human homologue of Gfi-1, a murine proto-oncogene, YY-1 and retinoic acid receptor.

Suppression of the murine CMVie by IFNγ and IFNγ has been partially attributed to cytokine-induced inhibition of NFκB activity. NFκB activity is inhibited by p202, which is induced by IFNγ and directly binds the p50 and p65 subunits of NFκB. Further, p202 can bind the c-fos and c-jun subunits of AP-1 which may have inhibitory effects. The IFNγ-induced Stat-1 protein competes for CBP and p300, which are co-activators for AP-1 and ETS transcription activity.

It has been speculated that sequences upstream from −285 nt could be involved in down regulation of the IE enhancer by IFN-α, Gribando et al., Virology 197: 303-311, 1993. In addition, International Patent Publication No. WO 97/48720 by Tsichlis et al., published Dec. 24, 1997, describes mutated promoter sequences which no longer bind the Gfi-1 transcriptional repressor.

In summary, the human cytomegalovirus (CMV) major immediate early promoter is widely used in current gene therapy protocols to express transgenes in vivo. A major limitation of using the CMV promoter is short-term, low-level transgene expression which is associated with promoter suppression. Cytokines such as IFNγ and TNFα which are elicited by gene therapy vectors have been shown to suppress transgene expression in vitro and in vivo.

### SUMMARY

The present invention relates generally to gene expression and regulation, and more particularly to cytokine resistant cytomegalovirus promoter mutants useful in gene therapy. Methods of making and using such promoters are also provided. Such products and methods will allow more consistent gene expresssion from patient to patient.

Thus, in one aspect, the invention features a modified cytomegalovirus promoter that enhances the duration or intensity of expression of a desired gene in the presence of one or more cytokines relative to the expression produced by the corresponding non-modified cytomegalovirus promoter in the presence of the one or more cytokines.

By “modified” is meant that a naturally occurring cytomegalovirus promoter has been changed in some way. For example, the nucleic acid sequence of the naturally occurring CMV promoter may be mutated by deletion, addition, or replacement of one or more nucleotides. The term “modified,” “modification,” “mutant” or “mutated” refers to an alteration of the promoter from its naturally occurring wild-type form. This includes alteration of the primary sequence of a promoter such that it differs from the wild-type or naturally-occurring sequence. The mutant promoter as used in the present invention can be a mutant of any member of the CMV promoter family.

One skilled in the art will recognize that a combination of mutations and/or deletions are possible to gain the desired response. This would include double point mutations to the same or different domains. In addition, mutation also includes “null mutations” which are genetic lesions to a gene locus that totally inactivate the gene product.

The term mutation also includes any other derivatives. The term “derivative” as used herein refers to a compound produced or modified from another compound of a similar structure. Such a derivative may be a “chemical derivative,” “fragment,” “variant,” “chimera,” or “hybrid” of the promoter. A derivative retains at least a portion of the function of the promoter which permits its utility in accordance with the present invention, for example cytokine resistant expression.

A derivative may comprise at least one “variant” oligonucleotide which either lacks one or more nucleic acids or contain additional or substituted nucleic acids relative to the native oligonucleotide. The variant may be derived from a naturally by appropriately modifying the sequence to add, remove, and/or to modify codons for one or more amino acids at one or more sites of the C-terminus, N-terminus, and/or within the native sequence. It is understood that such variants having added, substituted and/or additional nucleic acids retain one or more characterizing portions of the native oligonucleotide. A functional derivative comprising oligonucleotide with deleted, inserted and/or substituted nucleic acid residues may be prepared using standard techniques well-known to those of ordinary skill in the art.

A “chemical derivative” contains additional chemical moieties not normally a part of the oligonucleotide. Such moieties may improve the molecule's solubility, absorption, biological half life, and the like.

The term “modified” or “modification”, as explained above, refers to a change in the composition or structure of the compound or molecule. However, the activity of the derivative, modified compound, or molecule is retained, enhanced, or increased relative to the activity of the parent compound or molecule. This would include the change of one nucleic acid in the sequence of the nucleotide or the introduction of one or more non-naturally occurring nucleic acids or other compounds. This includes a change in a chemical body, a change in a hydrogen placement, or any type of chemical variation. In addition, “analog” as used herein refers to a compound that resembles another structure. Analog is not necessarily an isomer. The above are only examples and are not limiting.

By “duration” is meant the amount of time a desired gene is expressed as measured, for example in months, weeks, days, hours, minutes and/or seconds.

By “intensity” is meant the rate at which a desired gene is expressed, for example mass or volume divided by time.

By “expression” is meant production of the encoded product, preferably by transcription and translation of the desired gene or nucleic acid sequence.

The term “cytokines” is meant to refer to the conventionally recognized group of immunogenic proteins such as IL-2, IL-3, IL-4, IL-6, IL-7, IL-8, IL-12, IL-18, TNF-α, INF-α, IFN-α and IFN-γ.

By “desired gene” is meant to refer to any gene or nucleic acid sequence encoding a product desired by the individual using the modified CMV promoter. Examples of desired genes include CAT, and therapeutic agents (capable of at least partially reducing or preventing one or more symptoms of a disease) such as IL-2 and all other cytokines, as well as all intracellullar proteins (e.g., thymadine kinase).

By “cytomegalovirus promotor” is meant one or more sequences from a cytomegalovirus which are functional in eukaryotic cells as a transcriptional promoter and an upstream enhancer sequence, such as the major immediate early promoter as conventionally understood by those skilled in the art. A CMV promoter generally includes an upstream enhancer, a base 1 promoter, and a downstream exon 1 leader/intron in sequence. Scc, Stinski et al., “Regulation of transcription from the cytomegaloviruse major intermediate early promoter by cellular and viral proteins” in Multidisciplinary approach to understanding cytomegalovirus (S. Michelson and S. A. Plotkin, Eds., 1993) Elsevier Science Publishers, Amsterdam, and Stinski et al., “Regulation of human cytomegalovirus transcription” in Herpesvirus transcription and its control (E. K. Wagner, Ed. 1991) CRC Press, Boca Raton. Examples of such are known in the art. The enhancer sequence allows transcription to occur at a higher frequency from the associated promoter.

In preferred embodiments the modified cytomegalovirus promoter includes a nucleic acid sequence that is substantially similar to the nucleic acid sequence encoding the non-modified cytomegalovirus promoter. By substantially similar is meant that the two sequences are highly homologous and preferably are at least 90%, 950% or 99% identical using a conventional computer alignment program, such as the GCG (Wiseman, Genetics Care Group) Program. However, sequences that are only similar are also encompassed (i.e., 60%, 75%, and 80% identical).

In other preferred embodiments, the modified cytomegalovirus promoter lacks one or more response elements present in the non-modified cytomegalovirus promoter and which interfere(s) with maximal expression of the desired gene when in the presence of the one or more cytokines.

By “response element” is meant a portion of the sequence of a naturally occurring CMV promoter which is responsible for a particular response of the promoter to a stimulus. For example, a sequence responsible for inhibition of expression in response to the presence of one or more cytokines.

Preferably the modified cytomegalovirus promoter lacks a number (for example, any integer between 1 and 300 inclusive) of contiguous nucleic acids from the 3′ or 5′ end of the non-modified cytomegalovirus promoter although similar deletions from the internal section of the promoter sequence were also possible.

The cytokines preferably are inflammatory cytokines, such as TNF-α, IFN-γ, IFN-α, IL-12, IL-6, and IL-16 and the modified cytomegalovirus promoter enhances expression of the desired gene in vitro and/or in vivo. The modified cytomegalovirus promoter may enhance the duration of expression of the desired gene, preferably at least two-fold (more preferably at least a fold, most preferably at least 10 fold), and/or the amount of expression of the desired gene.

In a preferred embodiment, the modified cytomegalovirus promoter has the nucleic acid sequence of the modified cytomegalovirus promoter of plasmid pLC1001 [SEQ ID NO:1] or pLC0888 [SEQ ID NO:2].

In other preferred embodiments, the modified cytomegalovirus promoter is a modified human cytomegalovirus promoter, the modification is not a mutation in a binding site for a Gfi-1 transcription repressor, and/or the desired gene encodes a compound selected from the group consisting of IL-12, interferon alpha, and TNF alpha.

In other aspects, the modified cytomegalovirus promoter is made by a process including the step of specifically deleting one or more sequences from the non-modified cytomegalovirus promoter which interfere with maximal expression of the desired gene when in the presence of the one or more cytokines. The sequence or sequences to be deleted can be identified as explained hereon (using random mutagenesis and testing for activity or rational design and confirmation) and the actual deletion can be achieved by chemical synthesis of the shorter or otherwise modified sequence, or by any other conventional technique known in the art, such as PCR, random oligonucleotide synthesis, and various shuffling techniques.

Alternatively the process includes the steps of: (a) transfecting cells with a combinatorial library of modified cytomegalovirus promoters ligated into a green fluorescent protein expression vector, wherein the combinatorial library comprises a series of modified cytomegalovirus promoters, wherein each modified cytomegalovirus promoter in the series includes an independently modified form of a corresponding non-modified cytomegalovirus promoter; (b) culturing the transfected cells in the presence of one or more cytokines; (c) selecting GFP expressing cells by FACS sorting; and (d) isolating episomal DNA fraction from cells sorted for high GFP expression.

By “transfecting” is meant the delivery and expression of a gene to cells.

By “combinatorial library” is meant a series of modified cytomegalovirus promoters wherein each modified cytomegalovirus promoter in the series includes an independently modified form of a corresponding non-modified cytomegalovirus promoter.

A “green fluorescent protein expression vector” refers to the plasmid in which the combinatorial library is built. The vector includes a green fluorescent protein gene, such as the green fluorescent protein gene, isolated from the jellyfish Aequorea Victoria, encodes a protein that fluoresces upon excitation with blue-green light (Chalfie, Photochem Photobiol, 62:651, 1995; Chalfie et al., Science, 263:802, 1994). This gene is a useful reporter or marker since the protein when expressed intracellularly spontaneously fluoresces without added cofactors.

The term “high GFP expression” refers to GFP expression-duration as intensity greater than that produced using the corresponding non-modified CMV promoter under identical or substantially similar conditions.

In yet another aspect, the invention provides another method of making a modified cytomegalovirus promoter of the invention by chemically synthesizing the modified cytomegalovirus promoter. The synthesis may be performed using methodology and equipment known in the art. For example, with a commercially available synthetic gene constructed by ligation of oligonucleotides homo.logous to the gene.

Another aspect of the invention is a method of using a modified cytomegalovirus promoter of the invention by expressing the desired gene in the presence of the one or more cytokines. Thus, the invention provides an improved method of expressing a desired gene in the presence of one or more cytokines, the improvement including using a modified cytomegalovirus promoter of the invention.

Also provided is a vector or plasmid including a modified cytomegalovirus promoter of the invention and the desired gene.

By “vector” is meant a construction comprised of genetic material designed to direct transformation of a targeted cell. A vector contains multiple genetic elements positionally and sequentially oriented with other necessary elements such that the nucleic acid in a nucleic acid cassette can be transcribed and when necessary translated in the transfected cells. The term vector as used herein can refer to nucleic acid, e.g., DNA derived from a plasmid, cosmid, phagemid or bacteriophage, into which one or more fragments of nucleic acid may be inserted or cloned which encode for particular proteins.

The vector can contain one or more unique restriction sites, and may be capable of autonomous replication in a defined host or organism such that the cloned sequence is reproduced. The vector molecule can confer some well-defined phenotype on the host organism which is either selectable or readily detected. The vector may have a linear or circular configuration. The components of a vector can contain but is not limited to a DNA molecule incorporating: (1) DNA; (2) a sequence encoding a therapeutic or desired product; and (3) regulatory elements for transcription, translation, RNA processing, RNA stability, and replication.

The purpose of the vector is to provide expression of a nucleic acid sequence in cells or tissue. Expression includes the efficient transcription of an inserted gene or nucleic acid sequence. Expression products may be proteins, polypeptides, or RNA. The nucleic acid sequence can be contained in a nucleic acid cassette. Expression of the nucleic acid can be continuous, constitutive, or regulated. The vector can also be used as a prokaryotic element for replication of plasmid in bacteria and selection for maintenance of plasmid in bacteria.

In the present invention the preferred vector comprises the following elements linked sequentially at an appropriate distance to allow functional expression: a promoter, a 5′ mRNA leader sequence, a translation initiation site, a nucleic acid cassette containing the sequence to be expressed, a 3′ mRNA untranslated region, and a polyadenylation signal sequence. As used herein the term “expression vector” refers to a DNA vector that contains all of the information necessary to produce a recombinant protein in a heterologous cell.

In addition, the term “vector” as used herein can also include viral vectors. A “viral vector” in this sense is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a vector of the present invention, that portion does not cause incorporation of the vector into a viral particle and thus is unable to produce an infective viral particle.

A vector as used herein can also include DNA sequence elements which enable extra-chromosomal (episomal) replication of the DNA. Vectors capable of episomal replication are maintained as extra-chromosomal molecules and can replicate. These vectors are not eliminated by simple degradation but continue to be copied. These elements may be derived from a viral or mammalian genome. These provide prolonged or “persistent” expression as described below.

The term “persistent expression” as used herein refers to introduction of genes into the cell together with genetic elements which enable episomal (i.e., extrachromosomal) replication. This can lead to apparently stable transformation of the cell without the integration of the novel genetic material into the chromosome of the host cell.

“Stable expression” as used herein relates to the integration of genetic material into chromosomes of the targeted cell where it becomes a permanent component of the genetic material in that cell. Gene expression after stable integration can permanently alter the characteristics of the cell and its progeny arising by replication leading to stable transformation.

The term “plasmid” as used herein refers to a construction comprised of extrachromosomal genetic material, usually of a circular duplex of DNA which can replicate independently of chromosomal DNA. The plasmid does not necessarily replicate.

The term “plasmid” as noted above, refers to construct made up of genetic material (i.e., nucleic acids). It includes genetic elements arranged such that an inserted coding sequence can be transcribed in eukaryotic cells. Also, while the plasmid may include a sequence from a viral nucleic acid, such viral sequence does not cause the incorporation of the plasmid into a viral particle, and the plasmid is therefore a non-viral vector. Preferably a plasmid is a closed circular DNA molecule. The modified promoter may or optionally be transcriptionally linked to a coding region for a desired gene product.

In this context, “transcriptionally linked” means that in a system suitable for transcription, transcription will initiate under the direction of the control sequence(s) and proceed through sequences which are transcriptionally linked with that control sequences(s). Preferably no mutation is created in the resulting transcript, which would alter the resulting translation product.

The term “coding region” or “coding sequence” refers to a nucleic acid sequence which encodes a particular gene product for which expression is desired, according to the normal base pairing and codon usage relationships. Thus, the coding sequence must be placed in such relationship to transcriptional control sequences (possibly including control elements and translational initiation and termination codons) that a proper length transcript will be produced and will result in translation in the appropriate reading frame to produce a functional desired product.

In yet another aspect, the invention features a method of making a vector or plasmid including a modified cytomegalovirus promoter of the invention and the desired gene by combining the modified cytomegalovirus promoter and the desired gene. The promoter and gene may be combined, for example, by transcriptionally linking them together, as described above.

Another aspect of the invention provides a method of using a vector or plasmid including a modified cytomegalovirus promoter of the invention and the desired gene by expressing the desired gene in the presence of the one or more cytokines.

A combinatorial library of modified cytomegalovirus promoters is also provided and includes a series of modified cytomegalovirus promoters wherein each modified cytomegalovirus promoter in the series includes an independently modified form of a corresponding non-modified cytomegalovirus promoter.

In addition, a method of making a combinatorial library of the invention is provided and involves making a series of modified cytomegalovirus promoters (each one of which is made as described above). Each modified cytomegalovirus promoter in the series includes an independently modified form of a corresponding non-modified cytomegalovirus promoter.

In yet another aspect, the invention features a method of using a combinatorial library of the invention. The method involves exposing each member of the series to one or more test agents.

The term “test agents” refers to small organic chemical compounds, proteins or peptides, such as pro-inflamatory cytokines (i.e. TNF-α, IFN-γ, IFN-α, IL-8, IL-6, IL-1, etc.).

The invention also provides a method of screening modified cytomegalovirus promoters for those that enhance the duration or intensity of expression of a desired gene in the presence of one or more cytokines relative to the expression produced by the corresponding non-modified cytomegalovirus promoter in the presence of the one or more cytokines. The method involves using the modified cytomegalovirus promoter to express the desired gene in the presence of the one or more cytokines and comparing the expression to that produced using the non-modified promoter in the presence of the one or more cytokines. Those modified promoters which enhance expression, preferably to a significant amount (e.g., 2, 9 or 10 fold), can be selected for use.

In a preferred embodiment, the method involves the step of using a combinatorial library of modified cytomegalovirus promoters to express the desired gene in the presence of the one or more cytokines and comparing the expression to that produced using the non-modified promoter in the presence of the one or more cytokines. In this embodiment, the combinatorial library includes a series of modified cytomegalovirus promoters, and each modified cytomegalovirus promoter in the series includes an independently modified form of a corresponding non-modified cytomegalovirus promoter. The screening preferably involves FACS/MACS cell selection.

By “MACS cell selection is meant magnetic cell seperation, as is conventionall performed by those skilled in the art.

In another aspect the invention provides a method of treating or preventing a disease using a vector or plasmid of the invention.

By “treating or preventing” is meant causing at least temporary relief or improvement in a symptom or indication of a disease or condition. Thus, the amount is also sufficient to cause a pharmacological effect. The amount of the composition need not cause permanent improvement or improvement of all symptoms or indications. In one embodiment, the plasmid or vector includes a prostaglandin synthase gene. A therapeutically effective amount of a cancer therapeutic would be one that reduces overall tumor burden in the case of metastatic disease (i.e., the number of metasteses or their size) or one that reduces the mass of the tumor in localized cancers.

The condition or disease preferably is a cancer, such as epithelial glandular cancer, including adenoma and adenocarcinoma; squamous and transitional cancer, including polyp, papilloma, squamous cell and transitional cell carcinoma; connective tissue cancer, including tissue type positive, sarcoma and other (oma's); hematopoietic and lymphoreticular cancer, including lymphoma, leukemia and Hodgkin's disease; neural tissue cancer, including neuroma, sarcoma, neurofibroma and blastoma; mixed tissues of origin cancer, including teratoma and teratocarcinoma. Other cancerous conditions that are applicable to treatment include cancer of any of the following: adrenal gland, anus, bile duct, bladder, brain tumors: adult, breast, cancer of an unknown primary site, carcinoids of the gastrointestinal tract, cervix, childhood cancers, colon and rectum, esophagus, gall bladder, head and neck, islet cell and other pancreatic carcinomas, Kaposi's sarcoma, kidney, leukemia, liver, lung: non-small cell, lung: small cell, lymphoma: AIDS-associated, lymphoma: Hodgkin's disease, Lymphomas: non-Hodgkin's disease, melanoma, mesothelioma, metastatic cancer, multiple myeloma, ovary, ovarian germ cell tumors, pancreas, parathyroid, penis, pituitary, prostate, sarcomas of bone and soft tissue, skin, small intestine, stomach, testis, thymus, thyroid, trophoblastic disease, uterus: endometrial carcinoma, uterus: uterine sarcomas, vagina, or vulva. The composition preferably is administered by injection, although the method may also be performed ex vivo.

The vector or plasmid may be delivered using any of the techniques described in detail below, such as injection, inhalation, etc., either with or without a delivery system, such as a cationic lipid, a peptide-formulated plasmid, and/or a PINC delivery system.

In another aspect, the invention provides a formulation containing a vector or plasmid of the invention and a transfection facilitating agent. The transfection facilitating agent preferably is a protective/interactive non-condensing compound and the delivery preferably is to the cells of a mammal, for example by a needle-free injection device or by a pulse-voltage delivery device (such as an electroporation devive). Methods of making such formulations by combining the vector or plasmid and the transfection facilitating agent are also provided.

The term “transfection facilitating agent” as used herein refers to an agent that forms a complex with the nucleic acid. This molecular complex is associated with nucleic acid molecule in either a covalent or a non-covalent manner. The transfection facilitating agent should be capable of transporting nucleic acid molecules in a stable state and of releasing the bound nucleic acid molecules into the cellular interior. One of ordinary skill in the art could use well known DNA extraction methods, methods of immunofluorescence, or well known reporter gene methods such as for example CAT, or LacZ containing plasmids, in order to determine the transfection efficiency. The transfection facilitating agent should also be capable of being bound to nucleic acid molecules and lyophilized or freeze dried and either rehydrated prior to pulse voltage delivery.

In addition, the transfection facilitating agent may prevent lysosomal degradation of the nucleic acid molecules by endosomal lysis. Furthermore, the transfection facilitating agent allows for efficient transport of the nucleic acid molecule through the cytoplasm of the cell to the nuclear membrane and into the nucleus and provide protection.

In a preferred embodiment transfection facilitating agents are non-condensing polymers, oils and surfactants. These may be suitable for use as compounds which prolong the localized bioavailability of a nucleic acid: polyvinylpyrrolidones; polyvinylalcohols; propylene glycols; polyethylene glycols; polyvinylacetates; poloxamers (Pluronics)(block copolymers of propylene oxide and ethylene oxide, relative amounts of the two subunits may vary in different poloxamers); poloxamines (Tetronics); ethylene vinyl acetates; celluloses, including salts of carboxymethylcelluloses, methylcelluloses, hydroxypropylcelluloses, hydroxypropylmethylcelluloses; salts of hyaluronates; salts of alginates; heteropolysaccharides (pectins); dextrans; chitosans; phosphatidylcholines (lecithins); miglyols; polylactic acid; polyhydroxybutyric acid. More preferably some of these compounds may be used as, and are considered protective, interactive, non-condensing compounds (PINC) and others as sustained release compounds, while some may be used in either manner under the respectively appropriate conditions.

In another embodiment cationic condensing agents such as cationic lipids, peptides, or lipopetides may associate with the nucleic acid molecule and may facilitate transfection after pulse voltage delivery.

The PINC enhances the delivery of the nucleic acid molecule to mammalian cells in vivo, and preferably the nucleic acid molecule includes a coding sequence for a gene product to be expressed in the cell. In many cases, the relevant gene product is a polypeptide or protein. Preferably the PINC is used under conditions so that the PINC does not form a gel, or so that no gel form is present at the time of administration at about 30-40° C. Thus, in these compositions, the PINC is present at a concentration of 30% (w/v) or less. In certain preferred embodiments, the PINC concentration is still less, for example, 20% or less, 10% or less, 5% or less, or 1% or less. Thus, these compositions differ in compound concentration and functional effect from uses of these or similar compounds in which the compounds are used at higher concentrations, for example in the ethylene glycol mediated transfection of plant protoplasts, or the formation of gels for drug or nucleic acid delivery. In general, the PINCs are not in gel form in the conditions in which they are used as PINCs, though certain of the compounds may form gels under some conditions.

In connection with the protective, interactive, non-condensing compounds for these compositions, the term “non-condensing” means that an associated nucleic acid is not condensed or collapsed by the interaction with the PINC at the concentrations used in the compositions. Thus, the PINCs differ in type and/or use concentration from such condensing polymers. Examples of commonly used condensing polymers include polylysine, and cascade polymers (spherical polycations).

The term “protects” or “protective” or “protected” as used herein refers to an effect of the interaction between such a compound and a nucleic acid such that the rate of degradation of the nucleic acid is decreased in a particular environment, thereby prolonging the localized bioavailability of the nucleic acid molecule. Such degradation may be due a variety of different of factors, which specifically include the enzymatic action of a nuclease. The protective action may be provided in different ways, for example, by exclusion of the nuclease molecules or by exclusion of water.

The term “interactive” as used herein refers to the interaction between PINC's and nucleic acid molecules and/or cell wall components. Preferably, PINC polymers are capable of directly interacting with moieties of nucleic acid molecules and/or cell wall components. These interactions can facilitate transfection by, for example, helping associate the nucleic acid molecule-PINC complex closely with the cell wall as a result of biochemical interactions between the PINC and the cell wall and thereby mediate transfection. These interactions may also provide protection from nucleases by closely associating with the nucleic acid molecule.

Also in connection with such compounds and an associated nucleic acid molecule, the term “enhances the delivery” means that at least in conditions such that the amounts of PINC and nucleic acid is optimized, a greater biological effect is obtained than with the delivery of nucleic acid in saline. Thus, in cases where the expression of a gene product encoded by the nucleic acid is desired, the level of expression obtained with the PINC:nucleic acid composition is greater than the expression obtained with the same quantity of nucleic acid in saline for delivery by a method appropriate for the particular PINC/coding sequence combination.

In preferred embodiments of the above compositions, the PINC is polyvinyl pyrrolidone (PVP), polyvinyl alcohol (PVA), a PVP-PVA co-polymer, N-methyl-2-pyrrolidone (NM2P), ethylene glycol, or propylene glycol. In compositions in which a Poloxamer (Pluronics) is used, the nucleic acid is preferably not a viral vector, i.e., the nucleic acid is a non-viral vector.

In other preferred embodiments, the PINC is bound with a targeting ligand. Such targeting ligands can be of a variety of different types, including but not limited to galactosyl, residues, fucosal residues, mannosyl residues, carntitine derivatives, monoclonal antibodies, polyclonal antibodies, peptide ligands, and DNA-binding proteins. The targeting ligands may bind with receptors on cells such as antigen-presenting cells, hepatocytes, myocytes, epithelial cells, endothelial cells, and cancer cells.

In connection with the association of a targeting ligand and a PINC, the term “bound with” means that the parts have an interaction with each other such that the physical association is thermodynamically favored, representing at least a local minimum in the free energy function for that association. Such interaction may involve covalent binding, or non-covalent interactions such as ionic, hydrogen bonding, van der Waals interactions, hydrophobic interactions, and combinations of such interactions.

While the targeting ligand may be of various types, in one embodiment the ligand is an antibody. Both monoclonal antibodies and polyclonal antibodies may be utilized.

The nucleic acid may also be present in various forms. Preferably the nucleic acid is not associated with a compounds(s) which alter the physical form, however, in other embodiments the nucleic acid is condensed (such as with a condensing polymer), formulated with cationic lipids, formulated with peptides, or formulated with cationic polymers.

In preferred embodiments, the protective, interactive non-condensing compound is polyvinyl pyrrolidone, and/or the plasmid is in a solution having between 0.5% and 50% PVP, more preferably about 5% PVP. The DNA preferably is at least about 80% supercoiled, more preferably at least about 90% supercoiled, and most preferably at least about 95% supercoiled.

In another aspect the invention features a composition containing a protective, interactive non-condensing compound and a plasmid containing a modified cytomegalovirus promoter as described herein. In yet another aspect, the invention provides a PINC formulation of the invention as described above and a cationic lipid with a neutral co-lipid.

Preferably the cationic lipid is DOTMA and the neutral co-lipid is cholesterol (chol). DOTMA is 1,2-di-O-octadecenyl-3-trimethylammonium propane, which is described and discussed in Eppstein et al., U.S. Pat. No. 4,897,355, issued Jan. 30, 1990, which is incorporated herein by reference. However, other lipids and lipid combinations may be used in other embodiments. A variety of such lipids are described in Gao & Huang, 1995, Gene Therapy 2:710-722, which is hereby incorporated by reference.

As the charge ratio of the cationic lipid and the DNA is also a significant factor, in preferred embodiments the DNA and the cationic lipid are present is such amounts that the negative to positive charge ratio is about 1:3. While preferable, it is not necessary that the ratio be 1:3. Thus, preferably the charge ratio for the compositions is between about 1:1 and 1:10, more preferably between about 1:2 and 1:5.

The term “cationic lipid” refers to a lipid which has a net positive charge at physiological pH, and preferably carries no negative charges at such pH. An example of such a lipid is DOTMA. Similarly, “neutral co-lipid” refers to a lipid which has is usually uncharged at physiological pH. An example of such a lipid is cholesterol.

Thus, “negative to positive charge ratio” for the DNA and cationic lipid refers to the ratio between the net negative charges on the DNA compared to the net positive charges on the cationic lipid.

As the form of the DNA affects the expression efficiency, the DNA preferably is at least about 80% supercoiled, more preferably at least about 90% supercoiled, and most preferably at least about 95% supercoiled. The composition preferably includes an isotonic carbohydrate solution, such as an isotonic carbohybrate solution that consists essentially of about 10% lactose. In preferred embodiments, the composition the cationic lipid and the neutral co-lipid are prepared as a liposome having an extrusion size of about 800 nanometers. Preferably the liposomes are prepared to have an average diameter of between about 20 and 800 nm, more preferably between about 50 and 400 nm, still more preferably between about 75 and 200 nm, and most preferably about 100 nm. Microfluidization is the preferred method of preparation of the liposomes.

The compounds which protect the nucleic acid and/or prolong the localized bioavailability of a nucleic acid may achieve one or more of the following effects, due to their physical, chemical or rheological properties: (1) Protect nucleic acid, for example plasmid DNA, from nucleases due to steric, viscosity, or other effects such as shearing; (2) increase the area of contact between nucleic acid, such as plasmid DNA, through extracellular matrices and over cellular membranes, into which the nucleic acid is to be taken up; (3) concentrate nucleic acid, such as plasmid DNA, at cell surfaces due to water exclusion; (4) indirectly facilitate uptake of nucleic acid, such as plasmid DNA, by disrupting cellular membranes due to osmotic, hydrophobic or lytic effects; (5) indirectly facilitate uptake of nucleic acids by allowing diffusion of protected nucleic acid chains through tissue at the administration site; and (6) indirectly facilitate uptake of nucleic acid molecules through pore, holes, openings in the cells formed as a result of the electroporation process.

By “prolonging the localized bioavailability of a nucleic acid” is meant that a nucleic acid when administered to an organism in a composition comprising such a compound will be available for uptake by cells for a longer period of time than if administered in a composition without such a compound, for example when administered in a saline solution. This increased availability of nucleic acid to cells could occur, for example, due to increased duration of contact between the composition containing the nucleic acid and a cell or due to protection of the nucleic acid from attack by nucleases. The compounds which prolong the localized bioavailability of a nucleic acid are suitable for internal administration.

By “suitable for internal administration” is meant that the compounds are suitable to be administered within the tissue of an organism, for example within a muscle or within a joint space, epidermally, intradermally or subcutaneously. Properties making a compound suitable for internal administration can include, for example, the absence of a high level of toxicity to the organism as a whole.

By “delivery” or “delivering” is meant transportation of nucleic acid molecules to desired cells or any cells. The nucleic acid molecules will be delivered to multiple cell lines, including the desired target. Delivery results in the nucleic acid molecules coming in contact with the cell surface, cell membrane, cell endosome, within the cell membrane, nucleus or within the nucleus, or any other desired area of the cell from which transfection can occur within a variety of cell lines which can include but are not limited to; tumor cells, epithelial cells, Langerhan cells, Langhans' cells, littoral cells, keratinocytes, dendritic cells, macrophage cells, kupffer cells, lymphocytes and lymph nodes. Preferably, the formulation is delivered to the cells by electroporation and the nucleic acid molecule component is not significantly sheared upon delivery

The term “immune response” as used herein refers to the mammalian natural defense mechanism which can occur when foreign material is internalized. The immune response can be a global immune response involving the immune system components in their entirety. Preferably the immune response results from the protein product encoded by the formulated nucleic acid molecule. The immune response can be, but is not limited to; antibody production, T-cell proliferation/differentiation, activation of cytotoxic T-lymphocytes, and/or activation of natural killer cells. Preferably the immune response is a humoral immune response. However, as noted above, in other situations the immune response, preferably, is a cytotoxic T-lymphocyte response.

The term “humoral immune response” refers to the production of antibodies in response to internalized foreign material. Preferably the foreign material is the protein product encoded by a formulated nucleic acid molecule internalized by injection with a needle free device.

The term “needle-free injection device” as used herein relates to an apparatus that is capable of injecting an aerosol through and/or to the skin of a mammal into the tissue by air and/or mechanical pressure. It is understood that conventional devices of this type are calibrated to allow one of ordinary skill in the art to select and/or adjust the desired injection depth and therefore it is expected that future devices that perform this function will also be calibrated in the same manner. It is also understood that devices of this type may have a needle which is only used to collect a solution which is subsequently aerosolized, and delivered by needle-free means. The type of injection device is not considered a limiting aspect of the present invention. The primary importance of a needle-free device is, in fact, the capability of the device to deliver an aerosol of formulated nucleic acid molecules through and/or to the skin of a mammal. The needle-free injection device can include, for example, a Gene Gun or a Needle-Less Injector as described in U.S. Pat. No. 5,480,381 or a powder delivery device such as in PCT WO/097/134652.

The term “pulse voltage device”, or “pulse voltage injection device” as used herein relates to an apparatus that is capable of causing or causes injection of nucleic acid molecules into the cells of an organism by emitting a localized pulse of electricity to the cells, thereby causing the cell membrane to destabilize and result in the formation of passageways or pores into the cell. It is understood that conventional devices of this type are calibrated to allow one of ordinary skill in the art to select and/or adjust the desired voltage amplitude and/or the duration of pulsed voltage and therefore it is expected that future devices that perform this function will also be calibrated in the same manner. The type of injection device is not considered a limiting aspect of the present invention. The primary importance of a pulse voltage device is, in fact, the capability of the device to deliver formulated nucleic acid molecules into the cells of an organism. The pulse voltage injection device can include, for example, an electroporetic apparatus as described in U.S. Pat. Nos. 5,439,440, 5,704,908 or U.S. Pat. No. 5,702,384 or as published in PCT WO 96/12520, PCT WO 96/12006, PCT WO 95/19805, and PCT WO 97/07826, all of which are incorporated herein by reference in their entirety.

The term “apparatus” as used herein relates to the set of components that upon combination allow the delivery of formulations of nucleic acid molecules and transfection facilitating agents into the cells of an organism by pulse voltage delivery methods.

Preferably, the apparatus is capable of being calibrated to allow selection of pulse voltage amplitude and duration.

The apparatus of the invention can be a combination of a syringe or syringes, various combinations of electrodes, devices which are useful for target selection by means such as optical fibers and video monitoring, and a generator for producing voltage pulses which can be calibrated for various voltage amplitudes, durations and cycles. The syringe can be of a variety of sizes and can be selected to inject formulations at different delivery depths such as to the skin of an organism such as a mammal, or through the skin.

The term “skin” refers to the outer covering of a mammal consisting of epidermal and dermal tissue and appendages such as sweat ducts and hair follicles. Skin can comprise the hair of a mammal in cases where the mammal has an epidermis which is covered by hair. In mammals which have enough hair to be considered fur or a pelt it is preferable to shave the hair, leaving primarily skin.

The term “organism” as used herein refers to common usage by one of ordinary skill in the art. The organism can include; micro-organisms ,such as yeast or bacteria, plants, birds, reptiles, fish or mammals. The organism can be a companion animal or a domestic animal. Preferably the organism is a mammal and is therefore any warm blooded organism. More preferably the mammal is a human.

The term “companion animal” as used herein refers to those animals traditionally treated as “pets” such as for example, dogs, cats, horses, birds, reptiles, mice, rabbits, hamsters, and the like.

The term “domestic animal” as used herein refers to those animals traditionally considered domesticated, where animals such as those considered “companion animals” are included along with animals such as, pigs, chickens, ducks, cows, goats, lambs, and the like.

The summary of the invention described above is non-limiting and other features and advantages of the invention will be apparent from the following detailed description of the preferred embodiments, as well as from the claims.

### BRIEF DESCRIPTION OF THE DRAWINGS

FIG. 1 shows that a truncated CMV promoter is resistant to inhibition by inflammatory cytokines.

FIG. 2 shows inhibition of transgene expression in in vitro transfected tumor cell lines by cytokines.

FIG. 3 illustrates strategies to construct combinatorial plasmid libraries.

FIG. 4 shows a ligation strategy for making a combinatorial library of the invention.

FIG. 5 shows screening of combinatorial libraries of plasmids using FACS/MACS cell selection.

FIG. 6 shows the nucleotides sequences of oligonucleotides 1-9.

FIG. 7 shows oligomer sequences for various transcription factor binding sites.

FIG. 8 shows repeated elements of the human CMV ie enchancer/promoter.

FIG. 9 shows repeated repressor elements of the human CMV ie enhancer/promoter.

FIG. 10 shows the truncated CMV enhancer/promoter of pLC1001.

FIG. 11 shows cytokine-mediated inhibition of transgene mRNA expression.

FIG. 12 shows sensitivity of detecting GFP-positive cells by FACS following in vitro transfection of pGFP in COS cells.

FIG. 13 shows FACS selection of GFP-expressing plasmid.

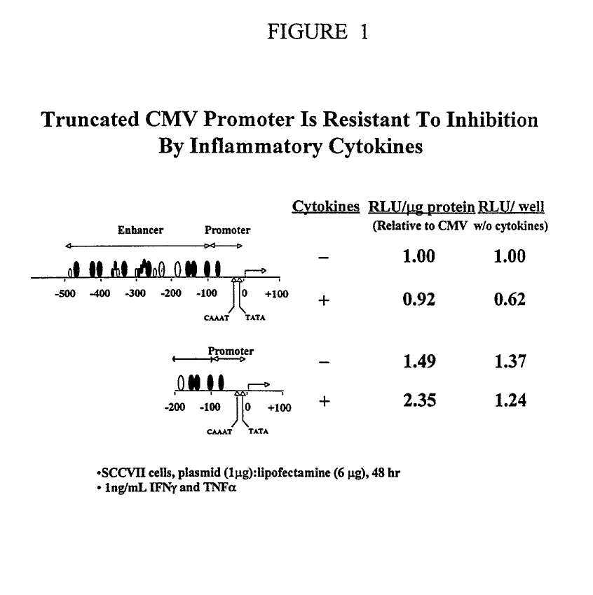

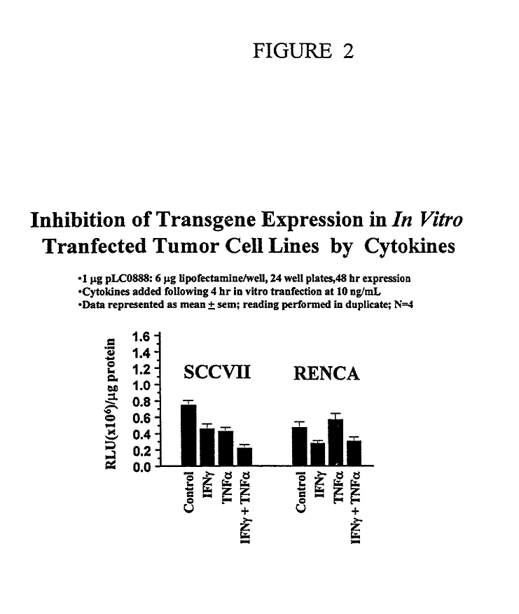

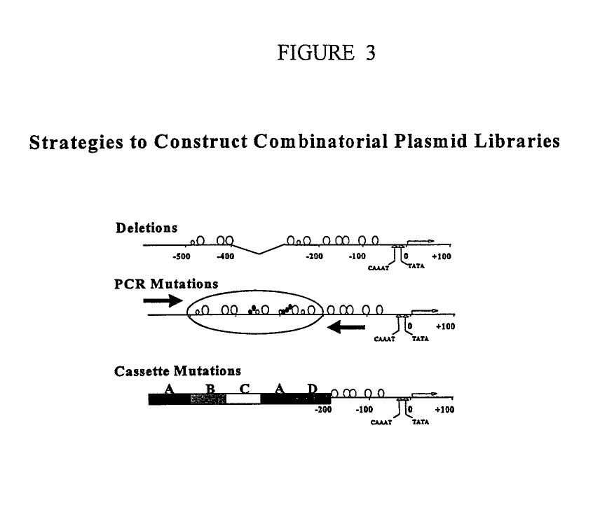

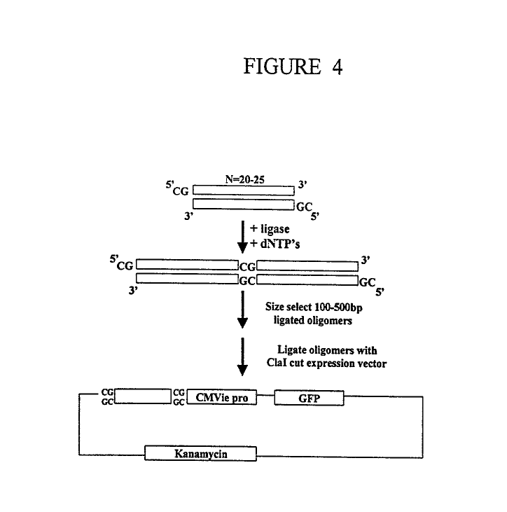

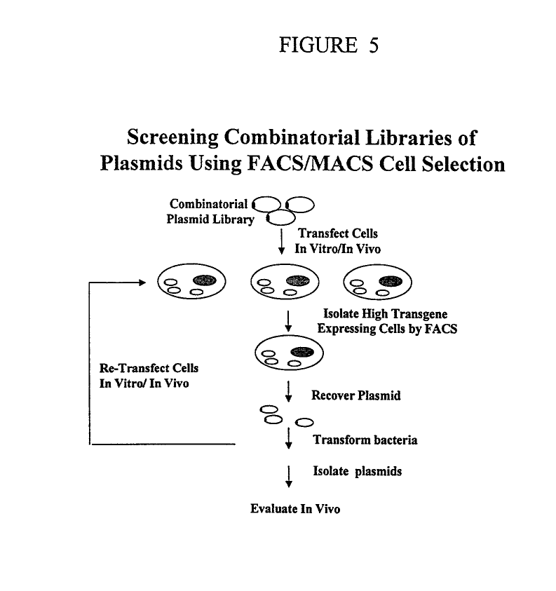

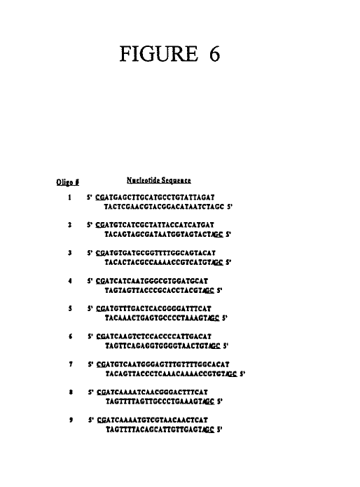

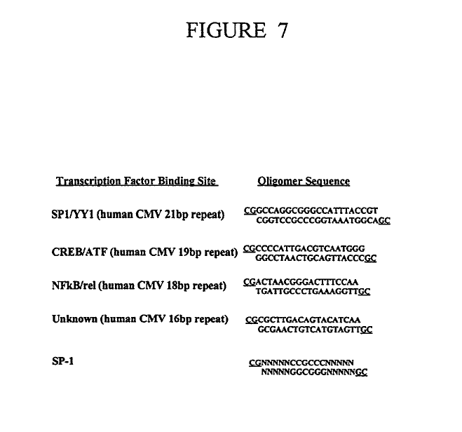

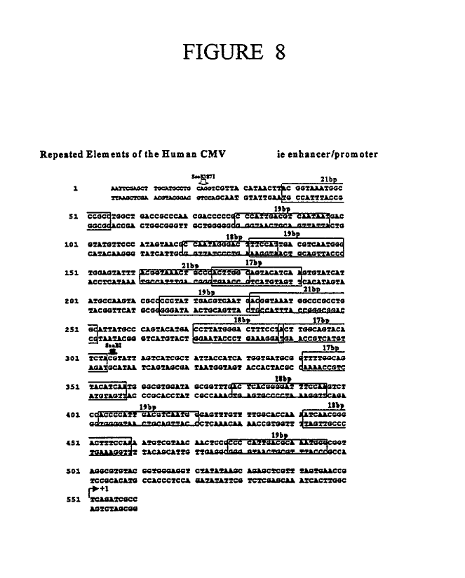

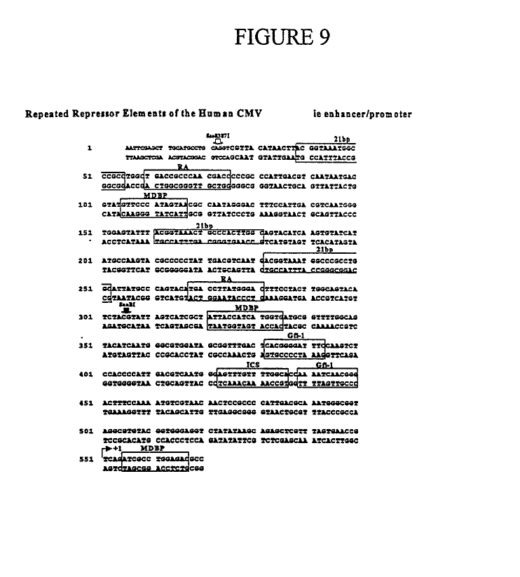

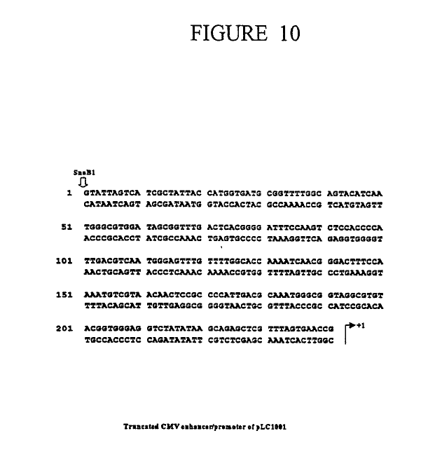

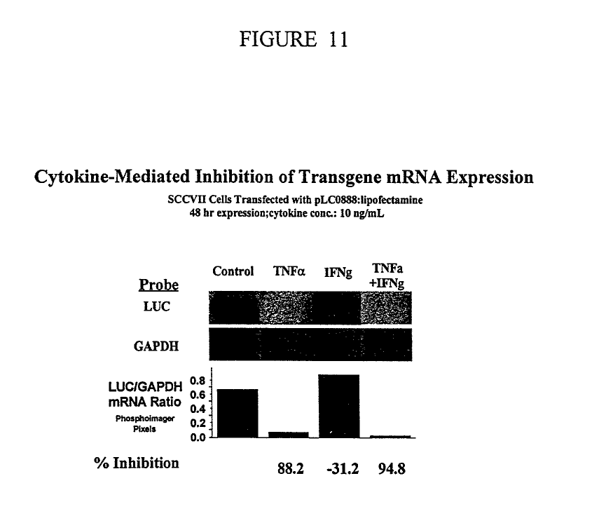

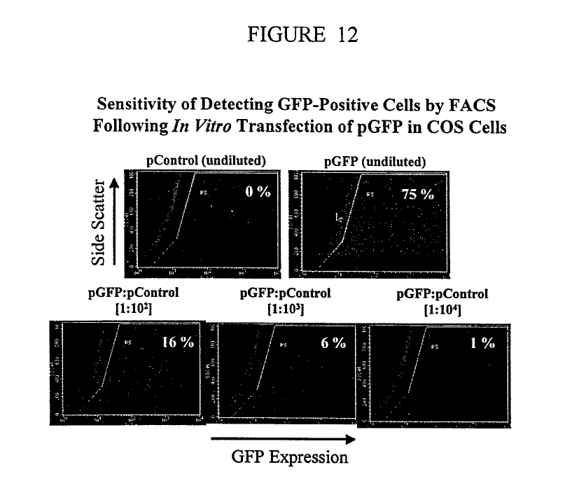

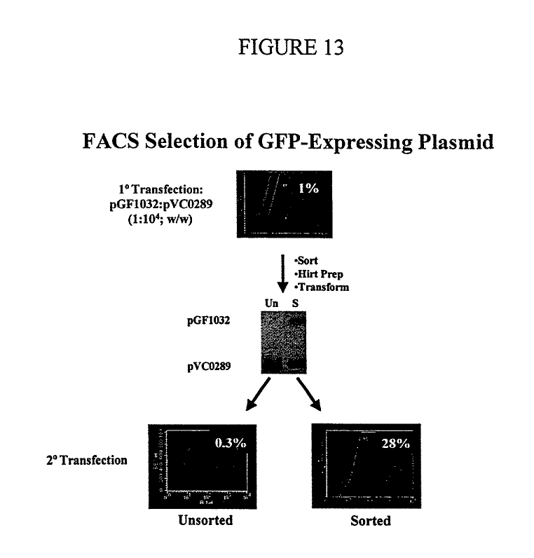

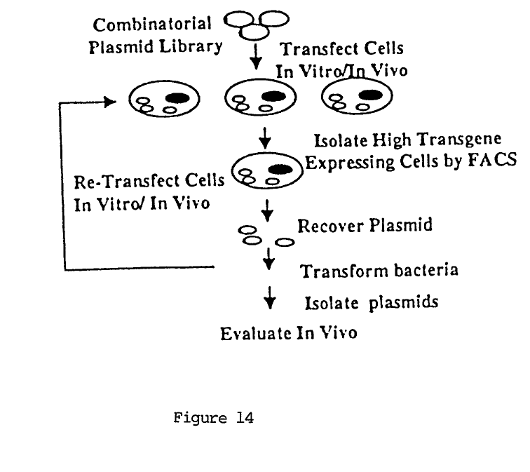

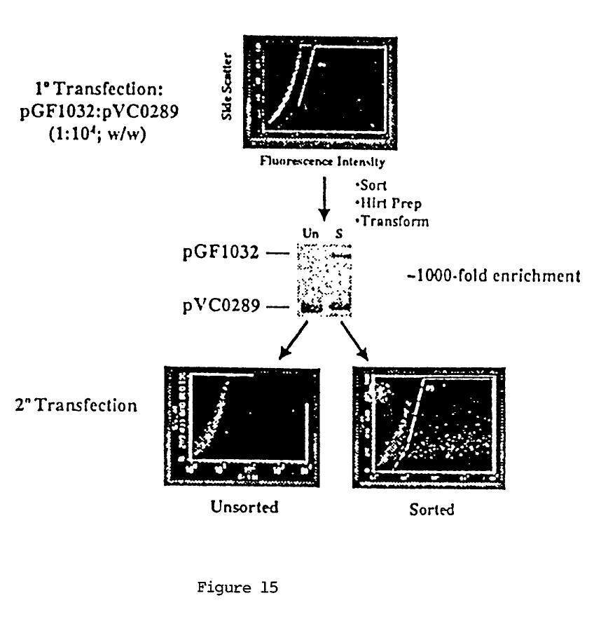

### DETAILED DESCRIPTION OF THE PREFERRED EMBODIMENTS

I. General

The present invention relates generally to gene expression and regulation, and more particularly to cytokine resistant cytomegalovirus promoter mutants useful in gene therapy. Methods of making and using such promoters are also provided. Such products and methods will provide more consistent transgene expression in vivo because expression will be independant of the effects of cytokines.

II. Selecting Target Modifications

Modifications to the CMV promoter which reduce the cytokine-induced suppression have been made using two methods. First, cells are transfected in vitro or in vivo with a combinatorial library of plasmids constructed with random synthetic oligonucleotides ligated upstream to a minimal CMV promoter and a green fluorescent protein (GFP) expression cassette. Selection of plasmids was performed by reiterative rounds of FACS (see. for example, Rice et al, Proc. Natl. Acad. Sci., USA, 89:5467-5471, 1992, incorporated herein by reference in its entirety, including any drawings) and episomal plasmid isolation from GFP-expressing cells cultured in the presence of IFNγ and TNFα.

In the second method, specific deletion of sequences within the native CMV enhancer leads to increased duration of promoter activity when transfected in cells in the presence of cytokines. In vivo administration of plasmids resistant to IFNγ/TNF60 promoter suppression improve the duration of transgene expression at least 2-fold. These modifications of the CMV promoter improve non-viral expression systems by reducing the number and dose of plasmid delivered.

Without wishing to be bound to any theory regarding the operation of the invention, it is noted that by removal or mutation of the negative transcription elements while maintaining or adding positive transcription elements, the duration and level of transgene expression following in vivo administration of CMV-based promoter plasmids will be enhanced.

There are a variety of factors which influence the effect of DNA sequence elements on promoter activity (such as position relative to the promoter, context in relation to other elements, and number of elements and orientation) that can be varied to optimize expression vectors for a given application.

A screening strategy to select from a combinatorial library of promoters ligated in an expression vector which allows the isolation of promoters which have sustained high-level transcriptional activity in the presence of cytokines is described below. The general approach is to: (a) transfect cells with a combinatorial promoter library ligated into a green fluorescent protein (GFP) expression vector, (b) culture transfected cells in the presence of cytokines, (c) select GFP expressing cells by FACS sorting, and (d) isolate episomal DNA fraction from cells sorted for high GFP expression. This method can be used to select expression plasmids following in vitro or preferably from in vivo transfected cells which are exposed to a more relevant cytokine-rich environment.

III. Making the Changes

A. Synthesis

Included within the scope of this invention are the functional equivalents of the herein-described isolated nucleic acid molecules. Functional equivalents or derivatives can be obtained in several ways.

The nucleic acid sequence may comprise a nucleotide sequence which results from the addition, deletion or substitution of at least one nucleotide to the 5′-end and/or the 3′-end of the non-modified nucleic acid or a derivative thereof. Any nucleotide or polynucleotide may be used in this regard, provided that its addition, deletion or substitution does not significantly alter the function of the promoter. For example, the present invention is intended to include any nucleic acid sequence resulting from the addition of ATG as an initiation codon at the 5′-end of an a nucleic acid sequence or its functional derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the CMV promoter genes and fragments thereof permitted by the genetic code are, therefore, included in this invention.

Further, it is possible to delete codons or to substitute one or more codons by codons other than degenerate codons to produce a structurally modified promoter, but one which has substantially the same utility or activity of the promoter produced by the unmodified nucleic acid molecule. As recognized in the art, the two promoters are functionally equivalent, even though the differences between the nucleic acid molecules are not related to degeneracy of the genetic code.

B. Nature of Wild-Type Promoter

The modified CMV promoter may be based on a corresponding non-modified promoter which has been isolated, pured or enriched.

By “isolated” in reference to nucleic acid is meant a polymer of 14, 17, 21 or more nucleotides conjugated to each other, including DNA or RNA that is isolated from a natural source or that is synthesized. The isolated nucleic acid of the present invention is unique in the sense that it is not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleotide sequence present, but that it is essentially free (about 90-95% pure at least) of non-nucleotide material naturally associated with it and thus is meant to be distinguished from isolated chromosomes.

By the use of the term “enriched” in reference to nucleic acid is meant that the specific DNA or RNA sequence constitutes a significantly higher fraction (2-5 fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it be noted that “enriched” does not imply that there are no other DNA or RNA sequences present, just that the relative amount of the sequence of interest has been significantly increased.

The term “significant” here is used to indicate that the level of increase is useful to the person making such an increase, and generally means an increase relative to other nucleic acids of about at least 2 fold, more preferably at least 5 to 10 fold or even more. The term also does not imply that there is no DNA or RNA from other sources. The other source DNA may, for example, comprise DNA from a yeast or bacterial genome, or a cloning vector such as pUC19. This term distinguishes the sequence from naturally occurring enrichment events, such as viral infection, or tumor type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does to require absolute purity such as a homogeneous preparation); instead, it represents an indication that the sequence is relatively purer than in the natural environment (compared to the natural level this level should be at least 2-5 fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus purification of at least one order of magnitude, preferably two or three orders, and more preferably four or five orders of magnitude is expressly contemplated. The term is also chosen to distinguish clones already in existence but which have not been isolated from other clones in a library of clones.

The term “nucleic acid molecule” describes a polymer of deoxyribonucleotides (DNA) or ribonucleotides (RNA). The nucleic acid molecule may be isolated from a natural source by cDNA cloning or subtractive hybridization or synthesized manually. The nucleic acid molecule may be synthesized manually by the triester synthetic method or by using an automated DNA synthesizer.

The term “cDNA cloning” refers to hybridizing a small nucleic acid molecule, a probe, to genomic cDNA. The probe hybridizes (binds) to complementary sequences of cDNA.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

IV. Testing the Modified Promoters

Modified promoters can be screened for those with appropriate activity using a variety of techniques known in the art, including those decribed herein, such as FACS/MACS screening an d selection. In particular, FACS is described in Rice et al., Proc. Natl. Acad. Sci., USA, 89:5467-5471, 1992, incorporated herein by reference in its entirety, including any drawings, and various MACS techniques are well known to those skilled in the art. An exemplary illustration of such techniques is provided in the examples below.

V. DNA Constructs Comprising a Modified CMV Promoter and Cells Containing These Constructs.

The present invention also relates to a recombinant DNA molecule comprising, 5′ to 3′, the promoter of the invention in a host cell and a desired nucleic acid molecule.

In addition, the present invention relates to a recombinant DNA molecules comprising a vector and a nucleic acid molecule described herein. The above-described molecules may be isolated and/or purified DNA molecules.

The present invention also relates to a cell or organism that contains modified CMV promoter linked to a coding sequence as described herein and thereby is capable of expressing a peptide. The polypeptide may be purified from cells which have been altered to express the polypeptide. A cell is said to be “altered to express a desired polypeptide” when the cell, through genetic manipulation, is made to produce a protein which it normally does not produce or which the cell normally provides at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but will in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a desired gene may be obtained by the above-described cloning methods. This region may be retained for its transcriptional termination regulatory sequences, such as termination and polyadenylation. Thus, by retaining the 3′-region naturally contiguous to the DNA sequence encoding a desired gene, the transcriptional termination signal may be provided. Where the transcriptional termination signals are not satisfactorily functional in the expression host cell, then a 3′ region functional in the host cell may be substituted.

Two DNA sequences (such as a promoter region sequence and a desired sequence). are said to be operably linked in the nature of the linkage between the two DNA sequences does no (1) result in the introduction of a frame-shift mutation, (2) interfere with the ability of the promoter region sequence to direct the transcription of a desired gene sequence, or (3) interfere with the ability of the a desired gene sequence to be transcribed by the promoter region sequence. Thus, a promoter region would be operably linked to a DNA sequence if the promoter were capable of effecting transcription of that DNA sequence. Thus, to express a desired gene, transcriptional and translational signals recognized by an appropriate host are necessary.

The present invention encompasses the expression of a desired gene (or a functional derivative thereof) in either prokaryotic or eukaryotic cells. Prokaryotic hosts are, generally, very efficient and convenient for the production of recombinant proteins and are, therefore, one type of preferred expression system for a desired gene. Prokaryotes most frequently are represented by various trans of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include λgt10, λgt11 and the like; and suitable virus vectors may include pMAM-neo, pKRC and the like. Preferably, the selected vector of the present invention has the capacity to replicate in the selected host cell.

Recognized prokaryotic hosts include bacteria such a E. coli and those from general such as Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a desired gene (or a functional derivative thereof) in a prokaryotic cell, it is necessary to operably link a desired sequence to a functional prokaryotic promoter, such as the modified CMV promoter of the invention.

Proper expression in a prokaryotic cell also requires the presence of a ribosome binding site upstream of the gene sequence-encoding sequence. Such ribosome binding sites are disclosed, for example, by Gold et al. (Ann. Rev. Microbiol. 35:365-404, 1981). The selection of control sequences, expression vectors, transformation methods, and the like, are dependent on the type of host cell used to express the gene.

As used herein, “cell,” “cell line,” and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of a peptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO, 3T3 or CHO-K1, or cells of lymphoid origin (such as 32D cells) and their derivatives. Preferred mammalian host cells include SP2/0 and J558L, as well as neuroblastoma cell lines such as IMR 332 and PC12 which may provide better capacities for correct post-translational processing.

In addition, plant cells are also available as hosts, and control sequences compatible with plant cells are available, such as the cauliflower mosaic virus 35S and 19S, and opaline synthase promoter and polyadenylation signal sequences. Another preferred host is an insect cell, for example the Drosophila larvae. Using insect cells as hosts, the Drosophila alcohol dehydrogenase promoter can be used. Rubin, Science 240:1453-1459, 1988). Alternatively baculovirus vectors can be engineered to express large amounts of a desired peptide or protein in insects cells (Jasny, Science 238:1653, 1987); Miller et al., In: Genetic Engineering (1986), Setlow, J. K., et al., eds., Plenum, Vol. 8, pp. 277-297).

Any of a series of yeast gene sequence expression systems can be utilized which incorporate promoter and termination elements from the actively expressed gene sequences coding for glycolytic enzymes are produced in large quantities when years are grown in mediums rich in glucose. Known glycolytic gene sequences can also provide very efficient transcriptional control signals. Yeast provides substantial advantages in that it can also carry out post-translational peptide modifications. A number of recombinant DNA strategies exist which utilize strong promoter sequences and high copy number of plasmids which can be utilized for production of the desired proteins in yeast. Yeast recognizes leader sequences on cloned mammalian gene sequence products and secretes peptides bearing leader sequences (i.e., pre-peptides). For a mammalian host, several possible vector systems are available for the expression of desired genes.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Translation of eukaryotic mRNA is initiated at the codon which encodes the first methionine. For this reason, it is preferable to ensure that the linkage between a promoter and a DNA sequence which encodes a desired peptide or protein does not contain any intervening codons which are capable of encoding a methionine (i.e., AUG). The presence of such codons results either in a formation of a fusion protein (if the AUG codon is in the same reading frame as a desired coding sequence) or a frame-shift mutation (if the AUG codon is not in the same reading frame as a desired coding sequence).

A desired nucleic acid molecule and an operably linked promoter may be introduced into a recipient prokaryotic or eukaryotic cell either as a nonreplicating DNA (or RNA) molecule, which may either be a linear molecule or, more preferably, a closed covalent circular molecule (a plasmid). Since such molecules are incapable of autonomous replication, the expression of the gene may occur through the transient expression of the introduced sequence. Alternatively, permanent or stable expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of single chain binding protein mRNA. These elements may include splice signals, as well as transcription promoters, enhancers, and termination signals. cDNA expression vectors incorporating such elements include those described by Okayama, Molec. Cell. Bio. 3:280(1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColEl, pSC101, pACYC 184, pVX. Such plasmids are, for example, disclosed by Sambrook (cf. “Molecular Cloning: A Laboratory Manual,” second edition, edited by Sambrook, Fritsch, & Maniatis, Cold Spring Harbor Laboratory, (1989)). Bacillus plasmids include pCl94, pC221, pT127, and the like. Such plasmids are disclosed by Gryczan (In: The Molecular Biology of the Bacilli, Academic Press, NY (1982), pp. 307-329). Suitable Streptomyces plasmids include plJ101 (Kendall et al., J. Bacteriol. 169:4177-4183, 1987), and streptomyces bacteriophages such as fC31 (Chater et al., In: Sixth International Symposium on Actinomycetales Biology, Akademiai Kaido, Budapest, Hungary (1986), pp. 45-54). Pseudomonas plasmids are reviewed by John et al. (Rev._{—}Infect. Dis. 8:693-704, 1986), and Izaki (Jpn. J. Bacteriol. 33:729-742, 1978).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982); Broach, In: “The Molecular Biology of the Yeast Saccharomyces: Life Cycle and Inheritance,” Cold Spring Harbor Laboratory, Cold Spring Harbor, NY, p. 445-470 (1981); Broach, Cell 28:203-204, 1982); Bollon et al., J. Clin. Hematol. Oncol. 10:39-48, 1980); Maniatis, In: Cell Biology: A Comprehensive Treatise, Vol. 3, Gene Sequence Expression, Academic Press, NY, pp. 563-608 (1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the desired peptide or protein. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

VI. Use

A. Diseases to Be Treated.

The condition or disease preferably is a cancer, such as epithelial glandular cancer, including adenoma and adenocarcinoma; squamous and transitional cancer, including polyp, papilloma, squamous cell and transitional cell carcinoma; connective tissue cancer, including tissue type positive, sarcoma and other (oma's); hematopoietic and lymphoreticular cancer, including lymphoma, leukemia and Hodgkin's disease; neural tissue cancer, including neuroma, sarcoma, neurofibroma and blastoma; mixed tissues of origin cancer, including teratoma and teratocarcinoma. Other cancerous conditions that are applicable to treatment include cancer of any of the following: adrenal gland, anus, bile duct, bladder, brain tumors: adult, breast, cancer of an unknown primary site, carcinoids of the gastrointestinal tract, cervix, childhood cancers, colon and rectum, esophagus, gall bladder, head and neck, islet cell and other pancreatic carcinomas, Kaposi's sarcoma, kidney, leukemia, liver, lung: non-small cell, lung: small cell, lymphoma: AIDS-associated, lymphoma: Hodgkin's disease, Lymphomas: non-Hodgkin's disease, melanoma, mesothelioma, metastatic cancer, multiple myeloma, ovary, ovarian germ cell tumors, pancreas, parathyroid, penis, pituitary, prostate, sarcomas of bone and soft tissue, skin, small intestine, stomach, testis, thymus, thyroid, trophoblastic disease, uterus: endometrial carcinoma, uterus: uterine sarcomas, vagina, or vulva. The composition preferably is administered by injection, although the method may also be performed ex vivo.

B. Delivery and Routes of Administration.

Administration as used herein refers to the route of introduction of a plasmid or carrier of DNA into the body. In addition to the methods of delivery described above, the expression systems constructs and the delivery system formulations can be administered by a variety of different methods.

Administration can be directly to a target tissue or by targeted delivery to the target tissue after systemic administration. In particular, the present invention can be used for treating disease by administration of the expression system or formulation to the body in order to establishing controlled expression of any specific nucleic acid sequence within tissues at certain levels that are useful for gene therapy.

The preferred means for administration of vector (plasmid) and use of formulations for delivery are described above. The preferred embodiments are by direct injection using needle injection.

The route of administration of any selected vector construct will depend on the particular use for the expression vectors. In general, a specific formulation for each vector construct used will focus on vector uptake with regard to the particular targeted tissue, followed by demonstration of efficacy. Uptake studies will include uptake assays to evaluate cellular uptake of the vectors and expression of the DNA of choice. Such assays will also determine the localization of the target DNA after uptake, and establishing the requirements for maintenance of steady-state concentrations of expressed protein. Efficacy and cytotoxicity can then be tested. Toxicity will not only include cell viability but also cell function.

Muscle cells have the unique ability to take up DNA from the extracellular space after simple injection of DNA particles as a solution, suspension, or colloid into the muscle. Expression of DNA by this method can be sustained for several months.

Delivery of formulated DNA vectors involves incorporating DNA into macromolecular complexes that undergo endocytosis by the target cell. Such complexes may include lipids, proteins, carbohydrates, synthetic organic compounds, or inorganic compounds. Preferably, the complex includes DNA, a cationic lipid, and a neutral lipid in particular proportions. The characteristics of the complex formed with the vector (size, charge, surface characteristics, composition) determines the bioavailability of the vector within the body. Other elements of the formulation function as ligand which interact with specific receptors on the surface or interior of the cell. Other elements of the formulation function to enhance entry into the cell, release from the endosome, and entry into the nucleus.

Delivery can also be through use of DNA transporters. DNA transporters refers to molecules which bind to DNA vectors and are capable of being taken up by epidermal cells. DNA transporters contain a molecular complex capable of noncovalently binding to DNA and efficiently transporting the DNA through the cell membrane. It is preferable that the transporter also transport the DNA through the nuclear membrane. See, e.g., the following applications all of which (including drawings) are hereby incorporated by reference herein: (1) Woo et al., U.S. Ser. No. 07/855,389, entitled “A DNA Transporter System and Method of Use, filed Mar. 20, 1992, now abandoned; (2) Woo et al., PCT/US93/02725, International Publ. WO93/18759, entitled “A DNA Transporter System and Method of Use”, (designating the U.S. and other countries) filed Mar. 19, 1993; (3) continuation-in-part application by Woo et al., entitled “Nucleic Acid Transporter Systems and Methods of Use”, filed Dec. 14, 1993, U.S. Ser. No. 08/167,641; (4) Szoka et al. , U.S. Ser. No. 07/913,669, entitled “Self-Assembling Polynucleotide Delivery System”, filed Jul. 14, 1992 and (5) Szoka et al., PCT/US93/03406, International Publ. WO93/19768 entitled “Self-Assembling Polynucleotide Delivery System”, (designating the U.S. and other countries) filed Apr. 5, 1993.

A DNA transporter system can consist of particles containing several elements that are independently and non-covalently bound to DNA. Each element consists of a ligand which recognizes specific receptors or other functional groups such as a protein complexed with a cationic group that binds to DNA. Examples of cations which may be used are spermine, spermine derivatives, histone, cationic peptides and/or polylysine.

One element is capable of binding both to the DNA vector and to a cell surface receptor on the target cell. Examples of such elements are organic compounds which interact with the asialoglycoprotein receptor, the folate receptor, the mannose-6-phosphate receptor, or the carnitine receptor.

A second element is capable of binding both to the DNA vector and to a receptor on the nuclear membrane. The nuclear ligand is capable of recognizing and transporting a transporter system through a nuclear membrane. An example of such ligand is the nuclear targeting sequence from SV40 large T antigen or histone.

A third element is capable of binding to both the DNA vector and to elements which induce episomal lysis. Examples include inactivated virus particles such as adenovirus, peptides related to influenza virus hemagglutinin, or the GALA peptide described in the Szoka patent cited above.

Transfer of genes directly into a tumor using a modified promoter of the invention will be very effective. This means of transfer is a preferred embodiment.

Administration may also involve lipids as described in preferred embodiments above. The lipids may form liposomes which are hollow spherical vesicles composed of lipids arranged in unilamellar, bilamellar, or multilamellar fashion and an internal aqueous space for entrapping water soluble compounds, such as DNA, ranging in size from 0.05 to several microns in diameter. Lipids may be useful without forming liposomes. Specific examples include the use of cationic lipids and complexes containing DOPE which interact with DNA and with the membrane of the target cell to facilitate entry of DNA into the cell.

Gene delivery can also be performed by transplanting genetically engineered cells. For example, immature muscle cells called myoblasts may be used to carry genes into the muscle fibers. Myoblast genetically engineered to express recombinant human growth hormone can secrete the growth hormone into the animal's blood. Secretion of the incorporated gene can be sustained over periods up to 3 months.

Myoblasts eventually differentiate and fuse to existing muscle tissue. Because the cell is incorporated into an existing structure, it is not just tolerated but nurtured. Myoblasts can easily be obtained by taking muscle tissue from an individual who needs gene therapy and the genetically engineered cells can also be easily put back with out causing damage to the patient's muscle. Similarly, keratinocytes may be used to delivery genes to tissues. Large numbers of keratinocytes can be generated by cultivation of a small biopsy. The cultures can be prepared as stratified sheets and when grafted to humans, generate epidermis which continues to improve in histotypic quality over many years. The keratinocytes are genetically engineered while in culture by transfecting the keratinocytes with the appropriate vector. Although keratinocytes are separated from the circulation by the basement membrane dividing the epidermis from the dermis, human keratinocytes secrete into circulation the protein produced.

The chosen method of delivery should result in expression of the gene product encoded within the nucleic acid cassette at levels which exert an appropriate biological effect. The rate of expression will depend upon the disease, the pharmacokinetics of the vector and gene product, and the route of administration, but should be in the range 0.001-100 mg/kg of body weight/day, and preferably 0.01-10 mg/kg of body weight/day. This level is readily determinable by standard methods. It could be more or less depending on the optimal dosing. The duration of treatment will extend through the course of the disease symptoms, possibly continuously. The number of doses will depend upon the disease, delivery vehicle, and efficacy data from clinical trials.

C. Gene Therapy.

A variety of proteins or their genetic sequences, both mutated and non-mutated, will also be useful in gene therapy (reviewed in Miller, Nature 357:455-460, (1992). Miller states that advances have resulted in practical approaches to human gene therapy that have demonstrated positive initial results. The basic science of gene therapy is described in Mulligan, Science 260:926-931, (1993).

In one preferred embodiment, an expression vector containing coding sequence is inserted into cells, the cells are grown in vitro and then infused in large numbers into patients. In another preferred embodiment, a DNA segment containing a promoter of choice (for example a strong promoter) is transferred into cells containing an endogenous desired gene in such a manner that the promoter segment enhances expression of the endogenous desired gene (for example, the promoter segment is transferred to the cell such that it becomes directly linked to the endogenous desired gene).

The gene therapy may involve the use of an adenovirus containing desired gene cDNA targeted to an appropriate cell type, systemic desired gene increase by implantation of engineered cells, injection with desired gene virus, or injection of naked desired gene DNA into appropriate cells or tissues, for example neurons.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encoding recombinant protein into the targeted cell population (e.g., tumor cells or neurons). Methods which are well known to those skilled in the art can be used to construct recombinant viral vectors containing coding sequences. See, for example, the techniques described in Maniatis et al., Molecular cloning: A Laboratory Manual, Cold Spring Harbor Laboratory, N.Y. (1989), and in Ausubel et al., Current Protocols in Molecular Biology, Greene Publishing Associates and Wiley Interscience, N.Y. (1989). Alternatively, recombinant nucleic acid molecules encoding protein sequences can be used as naked DNA or in reconstituted system eg., liposomes or other lipid systems for delivery to target cells (see e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins. See, Miller, supra.

In its simplest form, gene transfer can be performed by simply injecting minute amounts of DNA into the nucleus of a cell, through a process of microinjection. (Capecchi MR, Cell 22:479-88), 1980). Once recombinant genes are introduced into a cell, they can be recognized by the cells normal mechanisms for transcription and translation, and a gene product will be expressed. Other methods have also been attempted for introducing DNA into larger numbers of cells. These methods include: transfection, wherein DNA is precipitated with CaPO₄ and taken into cells by pinocytosis (Chen C. and Okayama H, Mol. Cell Biol. 7:2745-52, 1987); electroporation, wherein cells are exposed to large voltage pulses to introduce holes into the membrane (Chu G., et al., Nucleic Acids Res., 15:1311-26, 1987); lipofection/liposome fusion, wherein DNA is packaged into lipophilic vesicles which fuse with a target cell (Felgner PL., et al., Proc. Natl. Acad. Sci. USA. 84:7413-7, 1987)); and particle bombardment using DNA bound to small projectiles (Yang N. S. et al., Proc. Natl. Acad. Sci., USA 87:9568-72, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene. Curiel D. T. et al., Am. J. Respir. Cell. Mol. Biol., 6:247-52, 1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cytoplasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a desired protein is provided in which the nucleic acid sequence is expressed only in specific tissue. Methods of achieving tissue-specific gene expression as set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

VII. Formulations for In Vivo Delivery

1. General

While expression systems such as those described above provide the potential for expression when delivered to an appropriate location, it is beneficial to provide the expression system construct(s) in a delivery system which can assist both the delivery and the cellular uptake of the construct. Thus, this invention also provides particular formulations which include one or more expression system constructs (e.g., DNA plasmids as described above), and a protective, interactive non-condensing compound.

An additional significant factor relating to the plasmid construct is the percentage of plasmids which are in a supercoiled (SC) form rather than the open circular (OC) form.

Molecular modeling has demonstrated that an exemplary PINC polymer, PVP, forms hydrogen bonds with the base pairs of a plasmid within its major groove and results in a hydrophobic surface on the plasmid due to the vinyl backbone of PVP. These interactions are supported by the modulation of plasmid zeta potential by PVP as well as by the inhibition of ethidium bromide intercalation into complexed plasmid. Apparent binding between PVP and plasmid has been correlated to pH and salt concentration and have demonstrated the effect of these parameters on β-gal expression after intramuscular injection of plasmid/PVP complexes [Mumper, R. J., et al., 1997. Submitted to Gene Therapy]. A summary of the physico-chemical properties of plasmid/PVP complexes is listed in Table I below.

TABLE I
Summary of the Physico-Chemical Properties of Plasmid/PVP Complexes

| Method | Result |
|---|---|
| Molecular modeling | Hydrogen bonding and hydrophobic |
| | plasmid surface observed |
| Fourier-transformed Infra-red | Hydrogen bonding demonstrated |
| DNase I challenge | Decreased rate of plasmid degradation in |
| | the presence of PVP |
| Microtitration Calorimetry | Positive heats of reaction indicative of an |
| | endothermic process |
| Potentiometric titration | One unit pH drop when plasmid and PVP |
| | are complexed |
| Dynamic Dialysis | Rate of diffusion of PVP reduced in the |
| | presence of plasmid |
| Zeta potential modulation | Surface charge of plasmid decreased by |
| | PVP |
| Ethidium bromide Inter- | Ethidium bromide intercalation reduced |
| calation complexation | by plasmid/PVP |
| Osmotic pressure | Hyper-osmotic formulation (i.e., |
| | 340 mOsm/kg H₂O) |
| Luminescence Spectroscopy | Plasmid/PVP binding decreased in salt |
| | and/or at pH 7 |

2. Histology of expression in muscle

Immunohistochemistry for β-gal using a slide scanning technology has revealed the uniform distribution of β-gal expression sites across the whole cross-sections of rat tibialis muscles. Very localized areas were stained positive for β-gal when CMV-β-gal plasmid was formulated in saline. β-gal positive cells were observed exclusively around the needle tract when plasmid was injected in saline. This is in agreement with previously published results [Wolff, J. A., et al., 1990, Science 247:1465-68; Davis, H. L., et al., 1993, Hum. Gene Ther. 4:151-9; Davis, H. L., et al., 1993, Hum. Gene Ther. 4:733-40].

In comparison, immunoreactivity for β-gal was observed in a wide area of muscle tissue after intramuscular injection of CMV-β-gal plasmid/PVP complex (1:17 w/w) in 150 mM NaCl. It appeared that the majority of positive muscle fibers were located at the edge of muscle bundles. Thus, staining for β-gal in rat muscle demonstrated that, using a plasmid/PVP complex, the number of muscle fibers stained positive for β-gal was approximately 8-fold greater than found using a saline formulation. Positively stained muscle fibers were also observed over a much larger area in the muscle tissue using the plasmid/PVP complex providing evidence that the injected plasmid was widely dispersed after intramuscular injection.

Thus, the enhanced plasmid distribution and expression in rat skeletal muscle was a result of both protection from extracellular nuclease degradation due to complexation and hyper-osmotic effects of the plasmid/PVP complex. However, Dowty and Wolff et al. have demonstrated that osmolarity, up to twice physiologic osmolarity, did not significantly effect gene expression in muscle [Dowty, M. E., and Wolff, J. A. In: J. A. Wolff (Ed.), 1994, Gene Therapeutics: Methods and Applications of Direct Gene Transfer. Birkhauser, Boston, pp. 82-98]. This suggests that the enhanced expression of plasmid due to PVP complexation is most likely due to nuclease protection and less to osmotic effects. Further, the surface modification of plasmids by PVP (e.g., increased hydrophobicity and decreased negative surface charge) may also facilitate the uptake of plasmids by muscle cells.

3. Structure-activity relationship of PINC polymers

There is a linear relationship between the structure of a series of co-polymers of vinyl pyrrolidone and vinyl acetate and the levels of gene expression in rat muscle. The substitution of some vinyl pyrrolidone monomers with vinyl acetate monomers in PVP resulted in a co-polymer with reduced ability to form hydrogen bonds with plasmids. The reduced interaction subsequently led to decreased levels of gene expression in rat muscle after intramuscular injection. The expression of β-gal decreased linearly (R=0.97) as the extent of vinyl pyrrolidone monomer (VPM) content in the co-polymers decreased.

These data demonstrate that pH and viscosity are not the most important parameters effecting delivery of plasmid to muscle cells since these values were equivalent for all complexes. These data suggest that enhanced binding of the PINC polymers to plasmid results in increased protection and bioavailability of plasmid in muscle.

4. Additional PINC systems

The structure-activity relationship described above can be used to design novel co-polymers that will also have enhanced interaction with plasmids. It is expected that there is “an interactive window of opportunity” whereby enhanced binding affinity of the PINC systems will result in a further enhancement of gene expression after their intramuscular injection due to more extensive protection of plasmids from nuclease degradation. It is expected that there will be an optimal interaction beyond which either condensation of plasmids will occur or “triplex” type formation, either of which can result in decreased bioavailability in muscle and consequently reduced gene expression.

As indicated above, the PINC compounds are generally amphiphilic compounds having both a hydrophobic portion and a hydrophilic portion. In many cases the hydrophilic portion is provided by a polar group. It is recognized in the art that such polar groups can be provided by groups such as, but not limited to, pyrrolidone, alcohol, acetate, amine or heterocyclic groups such as those shown on pp. 2-73 and 2-74 of CRC Handbook of Chemistry and Physics (72nd Edition), David R. Lide, editor, including pyrroles, pyrazoles, imidazoles, triazoles, dithiols, oxazoles, (iso)thiazoles, oxadiazoles, oxatriazoles, diaoxazoles, oxathioles, pyrones, dioxins, pyridines, pyridazines, pyrimidines, pyrazines, piperazines, (iso)oxazines, indoles, indazoles, carpazoles, and purines and derivatives of these groups, hereby incorporated by reference.

Compounds also contain hydrophobic groups which, in the case of a polymer, are typically contained in the backbone of the molecule, but which may also be part of a non-polymeric molecule. Examples of such hydrophobic backbone groups include, but are not limited to, vinyls, ethyls, acrylates, acrylamides, esters, celluloses, amides, hydrides, ethers, carbonates, phosphazenes, sulfones, propylenes, and derivatives of these groups. The polarity characteristics of various groups are quite well known to those skilled in the art as illustrated, for example, by discussions of polarity in any introductory organic chemistry textbook.

The ability of such molecules to interact with nucleic acids is also understood by those skilled in the art, and can be predicted by the use of computer programs which model such intermolecular interactions. Alternatively or in addition to such modeling, effective compounds can readily be identified using one or more of such tests as 1) determination of inhibition of the rate of nuclease digestion, 2) alteration of the zeta potential of the DNA, which indicates coating of DNA, 3) or inhibition of the ability of intercalating agents, such as ethidium bromide to intercalate with DNA.

5. Targeting Ligands

In addition to the nucleic acid/PINC complexes described above for delivery and expression of nucleic acid sequences, in particular embodiments it is also useful to provide a targeting ligand in order to preferentially obtain expression in particular tissues, cells, or cellular regions or compartments.

Such a targeted PINC complex includes a PINC system (monomeric or polymeric PINC compound) complexed to plasmid (or other nucleic acid molecule). The PINC system is covalently or non-covalently attached to (bound to) a targeting ligand (TL) which binds to receptors having an affinity for the ligand. Such receptors may be on the surface or within compartments of a cell. Such targeting provides enhanced uptake or intracellular trafficking of the nucleic acid.

The targeting ligand may include, but is not limited to, galactosyl residues, fucosal residues, mannosyl residues, carnitine derivatives, monoclonal antibodies, polyclonal antibodies, peptide ligands, and DNA-binding proteins. Examples of cells which may usefully be targeted include, but are not limited to, antigen-presenting cells, hepatocytes, myocytes, epithelial cells, endothelial cells, and cancer cells.

Formation of such a targeted complex is illustrated by the following example of covalently attached targeting ligand (TL) to PINC system:

TL PINC+Plasmid ----------->TL-PINC::::::Plasmid

Formation of such a targeted complex is also illustrated by the following example of non-covalently attached targeting ligand (TL) to PINC system

TL::::::PINC+Plasmid --------->TL::::::PINC::::::Plasmid or alternatively,

PINC+Plasmid ------------->PINC:::::::Plasmid+TL ------------>TL::::::PINC:::::::Plasmid

In these examples :::::::: is non-covalent interaction such as ionic, hydrogen-bonding, Van der Waals interaction, hydrophobic interaction, or combinations of such interactions.

A targeting method for cytotoxic agents is described in Subramanian et al., International Application No. PCT/US96/08852, International Publication No. WO 96/39124, hereby incorporated by reference. This application describes the use of polymer affinity systems for targeting cytotoxic materials using a two-step targeting method involving zip polymers, i.e., pairs of interacting polymers. An antibody attached to one of the interacting polymers binds to a cellular target. That polymer then acts as a target for a second polymer attached to a cytotoxic agent. As referenced in Subramanian et al., other two-step (or multi-step) systems for delivery of toxic agents are also described.

In another aspect, nucleic acid coding sequences can be delivered and expressed using a two-step targeting approach involving a non-natural target for a PINC system or PINC-targeting ligand complex. Thus, for example, a PINC-plasmid complex can target a binding pair member which is itself attached to a ligand which binds to a cellular target (e.g., a MAB). Binding pairs for certain of the compounds identified herein as PINC compounds as identified in Subramanian et al. Alternatively, the PINC can be complexed to a tareting ligand, such as an antibody. That antibody can be targeted to a non-natural target which binds to, for example, a second antibody.

Delivery and expression of nucleic acids in many formulations, such as in saline, is limited due to degradation of the nucleic acids by cellular components of organisms, such as for insance nucleases. Thus, protection of the nucleic acids when delivered in vivo can greatly enhance the resulting expression, and thereby enhance a desired pharmacological or therapeutic effect. It was found that certain types of compounds which interact with a nucleic acid (e.g., DNA) in solution but do not condense the nucleic acid provide in vivo protection to the nucleic acid, and correspondingly enhance the expression of an encoded gene product. Some of these compounds have been discussed in U.S. Pat. No. 08/484,777, filed Jun. 7, 1998, International Patent Application No. PCT/US96/05679 filed Apr. 23, 1996 and U.S. patent application Ser. No. 60/045,295, filed May 2, 1997 all of which are incorporated herein by reference in their entirety including any drawings.

The use of delivery systems designed to interact with plasmids and protect plasmids from rapid extracellular nuclease degradation is described in Mumper, R. J., et al., 1996, Pharm. Res. 13:701-709; and Mumper, R. J., et al., 1997. Submitted to Gene Therapy. A characteristic of the PINC systems is that they are non-condensing systems that allow the plasmid to maintain flexibility and diffuse freely throughout the muscle while being protected from nuclease degradation. While the PINC systems are primarily discussed below, it will be understood that cationic lipid based systems and systems utilizing both PINCS and cationic lipids are also within the scope of the present invention.

A common structural component of the PINC systems is that they are amphiphilic molecules, having both a hydrophilic and a hydrophobic portion. The hydrophilic portion of the PINC is meant to interact with plasmids by hydrogen bonding (via hydrogen bond acceptor or donor groups), Van der Waals interactions, or/and by ionic interactions. For example, PVP and N-methyl-2-pyrrolidone (NM2P) are hydrogen bond acceptors while PVA and PG are hydrogen bond donors.

All four molecules have been reported to form complexes with various (poly)anionic molecules [Buhler V., BASF Aktiengescellschaft Feinchemie, Ludwigshafen, pp 39-42; Galaev Y, et al., J. Chrom. A. 684:45-54 (1994); Tarantino R, et al. J. Pharm. Sci. 83:1213-1216 (1994); Zia, H., et al., Pharm. Res. 8:502-504 (1991);]. The hydrophobic portion of the PINC systems is designed to result in a coating on the plasmid rendering its surface more hydrophobic. Kabanov et al. have described previously the use of cationic polyvinyl derivatives for plasmid condensation designed to increase plasmid hydrophobicity, protect plasmid from nuclease degradation, and increase its affinity for biological membranes [Kabanov, A. V., and Kabanov, V. A., 1995, Bioconj. Chem. 6:7-20; Kabanov, A. V., et al., 1991, Biopolymers 31:1437-1443; Yaroslavov, A. A., et al., 1996, FEBS Letters 384:177-180].

Substantial protective effect is observed; up to at least a one log enhancement of gene expression in rat muscle over plasmid formulated in saline has been demonstrated with these exemplary PINC systems. The expression of reporter genes in muscle using plasmids complexed with the PINC systems was more reproducible than when the plasmid was formulated in saline. For example, the coefficient of variation for reporter gene expression in muscle using plasmid formulated in saline was 96±35% (n=20 studies; 8-12 muscles/study) whereas with coefficient of variation with plasmids complexed with PINC systems was 40±190 (n=30 studies; 8-12 muscles/study). The high coefficient of variation for reporter gene expression with plasmid formulated in saline has been described previously [Davis, H. L., et al., 1993, Hum. Gene Ther. 4:151-9]. In addition, in contrast with the results for DNA:saline, there was no significant difference in gene expression in muscle when plasmid with different topologies were complexed with polyvinyl pyrrolidone (PVP). This suggests that PVP is able to protect all forms of the plasmid from rapid nuclease degradation.

### EXAMPLES

The present invention will be more fully described in conjunction with the following specific examples which are not to be construed in any way as limiting the scope of the invention.

### Example 1

Construction and Testing of Cytokine Resistant CMV Promoter Materials

Materials and Methods

Constructional of Combinatorial Promoter/Enhancer

Plasmid Library

pGF1129 contains a bluescript backbone consisting of −100 to +1 of CMVie enhancer/promoter with a synthetic intron (IVS8), a NcoI/XbaI green fluorescence protein cDNA from pEGFP-NI (Clontech, Palo Alto, Calif.) and bovine growth hormone 3′UTR. PGF1187 contains a unique ClaI site inserted in the EcoRI site upstream of the promoter. Synthetic oligonucleotides (Genosys, The Woodlands, Tex.) were designed as random 20-mers either homologous to various transcription factor binding sites or as random oligimers with an additional 5′ AT overhang. PGF1187 was digested with ClaI (Promega), and the ends dephosphorulated with calf intestinal phosphatase. Synthetic oligonucleotides were ligated in varying ratios with T4 ligase and purified prior to ligation in to the dephosphorulated ClaI-digested PGF1187 vector. A typical library consists of ligating 1.0 mg of vector and 1.0 mg of pre-ligated oligomers. The ligation was transformed in to competent DH5a (E. coli) and plated on kanamycin/LB plates.

A fraction of the library was transformed to determine the colony number and size range of individual colonies. The remaining library was transformed and plated at 104 colonies per 100 mm LB/agar plate. Colonies were grown to ˜1 mm size, the bacterial ‘lawn’ scraped into a flask and amplified in 100 mL of LB/kanamycin. Plasmid was purified using conventional alkaline lysis methods (Qiagen).

In Vitro Transfection

Various mouse and human cell lines were grown in DMEM (GIBCO) supplemented with 100% FCS, sodium pyruvate and antibiotics (complete medium-CM). Cells were plated at 105 /mL in 10 mL CM in 100% (Corning, Cat# 25025). Twenty-fours after plating, cells were exposed to 10 mg plasmid: 60 mg lipofectamine (GIBCO-BRL) in 3.0 ml of DMEM containing no serum.

After 4 hours the medium was replaced with CM and cultured between 1-5 days with varying doses and combinations of mouse rec. TFNα, IFNγ (R&D Systems, Minneapolis, Minn.), or mouse IFNα (Lee Molecular Research, San Diego, Calif.). Cells were harvested from plates using 2.0 mL trypsin/EDTA solution and placed in a 15 mL conical tube. Eight mL of CM was added to tube and cells were pelleted by centrifugation at 100×g for 10 minutes at RT.

Flow Cytometry

Cells were harvested from tissue culture plates using 0.05% trypsin/0.5M EDTA (GIBCO BRL, Grand Island, N.Y.) and washed twice with PBS containing 2% FCS and resuspended at ˜107 cells/mL in PBS/2% FCS. To improve selection of live transfectants, cells were stained with propidium iodide (Molecular Probes, Eugene, Oreg.) was added at a concentration of 0.5 mg/mL. Forward scatter, side scatter and propidium iodide gates were placed to analyze and sort a uniform population of cells. Typically, transfected cells were aliquoted in 1.0 ml samples and kept on ice until use. Fluorescence analysis and sorts were performed using a FASCaliber (Becton-Dickinson, Sunnyvale, Calif.).

Episomal DNA Purification

Following FACS selection, cells were centrifuged at 100×g for 10 minutes at RT, resuspended in 1.0 mL PBS and transferred to a 1.5 mL microfuge tube. Tubes were briefly centrifuged to pellet cells and the supernatant discarded. Cells were tapped to dislodge cell pellet and gently resuspended in 0.4 mL of 0.6% SDS/10 mM EDTA and 0.1 mL of 5 M NaCl. Cells were kept on ice for 30 minutes, then centrifuged for 15 minutes at maximum speed at 40° C.

The supernatant was transferred to a fresh tube and extracted twice with 0.5 mL phenol:chloroform: isoamyl alcohol (25:24:1; v/v/v). The aqueous phase was mixed with 0.1 volumes of 3.0 M sodium acetate (pH 5.2), 1 volume of isopropanol, 10 mL of 2.0 mg/mL glycogen (5prime-3prime, Boulder, Colo.) and placed on dry ice for 30 minutes. Tubes were centrifuged for 30 minutes at maximum speed at 4° C., pellets washed with 0.25 mL 70% ETOH, briefly dried under vacuum and resuspended in 100 mL 10 mM Tris (pH 8.0) and 1 mM EDTA.

Southern and Northern Blot Analysis

For total RNA isolation, cells were resuspended in 1.0 mL of RNAsol and RNA was extracted using previously described methods. Total RNA was electrophoresed is a 1.5% FMC seakem GTG agarosein 2×mops gel for 4 hrs at 60 volts. RNA was transferred to nylon membranes and preblocked with Fisher UV Crosslinker Model #FB-UUXL-1000 (max crosslinking). Blots were hybridized with the following probes: 1) a 1656 bp NcoI/XbaI fragment of luciferase isolated from pLC0888; 2) a 454 bp fragment of mouse glyceraldehyde phosphate dehydrogenase. Probes were labelled with 32P-dCTP using the random primer method. Total DNA was isolated from cells using previously described techniques. DNA was digested with XbaI to linearized vector sequences and electrophoresed in 1% agarose/TBE gels at 60 volts for 4 hours.

DNA were stained with ethidium bromide to insure equal loading of wells, transferred to nylon membranes and preblocked with Fisher UV crosslinker. Blots were hybridized with 10⁷ cpm/mL (10 ml total) labelled probes for 2 hours (Amersham RapidHybe Buffer) at approximately 68° C. Blots were washed twice with 1×SSC, 1% SDS once with 0.1×SSC, 0.1% SDS, and exposed to phosphoimager cassettes for varying lengths of time. Probe specific bands were quantitated using a phosphoimager (Fuji, Japan).

Animals

Male C57BL/6 mice (20-25 g; Harlan Laboratories, Houston, Tex.) were maintained on ad libitum rodent feed and water at 23° C., humidity 40% and a 12 hr/12 hr light-dark cycle. Animals were acclimated for at least 3 days prior to the start of the study.

Effect of Cytokines on CMV Vector Transgene Expression

To determine whether the presence of cytokines could inhibit CMV promoter-directed transgene expression, SCCVII and RENCA mouse tumor cell lines were transfected with CMV-luciferase using lipofectamine and cultured for 48 hours in the presence of various combinations of cytokines.

The results indicate that the addition of IFNγ and/or TNFα to both the SCCVII and RENCA cell lines suppress luciferase protein levels compared to total protein levels.

In the SCCVII tumor cell line, the simultaneous addition of IFNγ and TNFα reduced the luciferase levels more than either cytokine alone. In comparison, reduced levels of luciferase in transfected RENCA cells responded only to IFNγ. The addition of both IFNγ and TFNα to transfected RENCA cells did further suppress luciferase levels compared to IFNγ alone.

Inhibition of Steady-state Transgene mRNA Levels by Cytokines

To determine the mechanism by which cytokines suppress transgene expression, total RNA isolated from cytokine-treated SCCVII cells transfected in vitro with pLC0888:lipofectamine complexes was subjected to northern blot analysis for steady-state levels of luciferase and GAPDH mRNA. From the same population of transfected cells, total DNA was isolated to determine the relative quantity of plasmid retained in cells.

Steady-state levels of luciferase mRNA decreased to 61.2% and 57.5% of control levels in cells incubated with IFNγ or TFNα, respectively. The steady-state level of transgene mRNA in cells incubated with both IFNγ and TFNα decreased to 29.8% of control levels. The presence of IFNγ or TFNα either alone or in combination does not alter the steady-state levels of GAPDH.

It is further shown by southern blot analysis that genomic DNA isolated from control and cytokine-treated cells contains the same relative quantities of plasmid that co-purifies with genomic DNA. These data indicate that cytokine-induced transgene expression is a result of decreased levels of specific transgene mRNA expression. It has not been determined whether the decreased transgene mRNA levels are due to lower transcriptional activity or an enhancement of transgene mRNA degradation.

Truncation of CMV Promoter Reduces Cytokine-induced Transgene Suppression

Based on the observation that cytokines reduce CMV vector transgene expression, it was hypothesized the CMV promoter contained genetic elements that either: i) bound repressing transcription factors upregulated by cytokine-induced signaling mechanisms, or ii) bound activating transcription factors that were inhibited by cytokine-induced signaling mechanisms.

A series of vectors were constructed by deletion of various regions within the CMVie enhancer. Deletion from Sse8387 to SnaBI site of the human CMVie promoter was used for construction of plasmid pLC1001, which has been identified to be resistant to cytokine-induced promoter shut-off when transfected in vitro in SCCVII cells. The mechanism of promoter shut-off has not been determined. The two 21 bp repeats that are removed in pLC1001 are possibly involved in cytokine-induced promoter shut-off.

The 21 bp repeat contains the YY-1 motif which has been shown to have a repressive effect on the human CMV promoter, Kothari, S., et al., Nucleic Acids Res. 19:1767, 1991; Lui, R., et al., Nuclei Acids Res. 22:2453, 1994.

It has also been observed that the addition of multimers of sequences that bind the MDBP DNA-binding site to a minimal human CMV promoter suppress transcriptional activity, Zhang, X., et al., Virology 182:865, 1991. Removal MDBP binding sites, where one motif has been removed in the pLC1001 construct, may improve the resistance to cytokine-induced promoter activity. Vectors which contain modifications of 21 bp repeat and MBDP can be constructed to determine the contribution of these motifs to the mechanism of reduced transgene mRNA levels.

Selection of Cytokine Resistant Promoters by FACS

One of the major challenges in modifying the human CMV promoter/enhancer to improve the strength or persistence of promoter activity in a cytokine-rich environment is to identify motifs with respect to orientation and context to adjacent DNA sequences. A method to identify plasmids by screening a combinatorial library of CMV promoter/enhancer constructs is provided here to help solve this problem.

Double-stranded oligonucleotides were synthesized with 5′-GC overhang to allow for random annealing to each other and to the ClaI insertion site in the vector pGF1187. Two sets of 20-25 mer oligonucleotides were designed as follows: i) 9 sets of double-stranded oligonucleotides having the homologous sequence contained in the pLC1001 CMV promoter/enhancer region extending from −246 to −50 relative to the start site.

These oligomers were chosen based on the resistance of pLC1001 to cytokine supplemented culture medium when transfected in vitro in SCCVII cells. ii) double-stranded oligonucleotides encoding various known sequences in the CMV promoter that encode DNA-binding protein motifs.

The strategy for construction of the library is now described. In brief, ligated and size-selected oligonucleotides are cloned into an expression vector containing a unique ClaI site upstream of a minimal human CMV promoter and a GFP expression cassette. The library is transformed into a suitable bacterial strain such as DH5a, plasmid is isolated from bacteria and transfected either in vitro or administered intratumorally in mice given a tumor cell line challenge. Transfected cells are subjected to FACS cell sorting to select cells with the 1-5% highest GFP fluorescence. Plasmid is purified from GFP-expressing cells, retransformed in to bacteria to amplify the plasmid copy number and subsequent retransfected in cells by either in vitro or in vivo methods.

After several reiterative rounds of FACS selection, plasmid isolation and in vitro/in vivo retransfection, a restricted set of plasmids are picked for individual analysis of activity in vitro and in vivo. A list of plasmids and the DNA sequence identified for improved transgene expression level and duration in a cytokine-rich environment is thus constructed.

### Example 2

Formulations: Gene Expression Systems and Gene Delivery Systems

Polymeric Gene Delivery System

The purified plasmids will be formulated as a complex with polyvinylpyrrolidone (PVP; Plasdone C-30, ISP Technologies, Wayne, N.J.), a polymer with a molecular weight of 50,000. A 5% bulk solution of PVP (pH 7.0) will be prepared following current Good Manufacturing Practices. The 5% PVP solution will be purified by 0.2 micron sterile filtration, vialed, lyophilized, and sealed under vacuum. Quality control testing is performed both on the 5% PVP solution and on the lyophilized PVP. Concentrated plasmid stock preparations will be obtained by lyophilizing and rehydrating plasmid with water to a final concentration of 3-5 mg/ml. The plasmid will be complexed to PVP at 1:17 w/w ratio and formulated in 150 mM NaCl as described (Mumper, et al, Pharm. Res. 13:701, 1996). The pH of the formulation will be pH 4 because the highest activity has been observed when the plasmid was formulated at that pH.

Cationic Gene Delivery System

The purified plasmids will be formulated as a complex with DOTMA (N-(1-(2-(3-dioleyloxy)propyl)-n-n-n-trimethylammonium chloride):Cholesterol [1:1 mole:mole, 400 nm extruded] at a 1:0.5 (−:+) charge ratio in 10% lactose. The mean diameter and zeta potential of the complexes will be characterized using dynamic light scattering and Doppler electrophoretic light scattering.

Construction of Combinatorial Promoter Libraries

A plasmid designated pGF1187 has been constructed which containing a minimal CMV promoter to which random olinucleotides are inserted at a unique restriction site immediately upstream of the promoter. pGF1187 was constructed using a bluescript (Strategene, San Diego, Calif.) backbone with the ampicillin resistence gene replaced with the kanamycin resistence gene. A synthetic minimal CMV promoter was inserted which includes the CAAT and TATA boxes from −73 to +6 of the transcription start.

A synthetic 5′ unstranslated region, termed IU58/UT12; as described in International Patent Application No. PCT/US97/18779, filed Oct. 10, 1997, entitled “IL-12 Gene Expression and Delivery Systems and Uses” by Bruce Freimark and Deepa Deshpande, incorporated herein by reference in its entirety, including any drawings, was placed between the promoter and the GFP gene. A NcoI/XbaI fragment encoding the GFP protein was excised from pEGFP-N1 (Clontech Laboratories, Inc., Palo Alto, Calif.) and subcloned into the unique NcoI/XbaI site of the plasmid backbone. The 3′UTR was derived from the bovine growth hormone gene. A unique ClaI has been placed immediately upstream of the minimal promoter and serves as the cloning site for insertion of the combinatorial oligonucleotides.

Oligonucleotides will be synthesized as 5′ phosphorulated 20-25 mers (Genosys, The Woodlands, Tex.) which are derived from the CMV enhancer sequence or specific transcription factor bindings sites with an additional 5′ CG dinucleotide added to the sequence. Both the positive and negative strands will be synthesized and annealed together by heat denaturation at 80° C. for 15 minutes and cooling for 30 minutes at room temperature. Double-stranded oligonucleotide pairs will be ligated together using T4 ligase (Promega, Madison, Wis.).

Ligated oligonucleotides will be electrophoresed on 1.5% agarose/TBE gel and the 200-600 bp fragments are purified by excising the agarose plug using glass beads (Geneclean, Bio101, Vista, Calif.). pGF1187 plasmid will be grown in a dam-strain of E. coli to ensure complete digestion with ClaI. ClaI-digested pGF1187 will be treated with calf intestinal phosphatase to minimize religation. Size-selected oligonucleotides will be ligated into the prepared plasmid and electroporated into bacteria (DH10a Electromax, BRL, Gaitherburg, Md.) by electroporation using standard conditions. Libraries will be plated on LB/kanamycin media and grown'overnight at 37EC. Individual colonies will be pooled, grown in liquid culture and the plasmid purified by alkaline lysis (Qiagen, Inc., Santa Clarita, Calif.).

Transfection and Selection

Cells will be seeded at 10⁵ cells/mL in 100 mm Petri dishes 24 hours prior to transfection. Cells will be transfected with 10 μg of plasmid library and 60 ug of lipofectamine for 4 hours in 3.0 ml serum-free RPMI-1640 containing penicillin/streptomycin and glutamine. Four hours following exposure, the medium will be replaced with 10 mL of complete medium with or without various combinations of cytokines. Forty-eight hours after transfection, the cells will be removed from the plates using 0.5% tryspin/EDTA. Cells will be resuspended in 2% FCS-DPBS and subjected to selection using a Becton-Dickinson FACS Caliber with 510 nm band pass filter for optimum GFP detection. Dead cells will be gated out by staining cells with propidium iodide and detected with a 640 nm band pass filter.

The 1-5% brightest cell population will be sorted. Plasmid recovery will be performed by isolation of episomal low molecular weight DNA. The recovered plasmid will be re-transformed into DH10α E. coli by electroporation and plated on 100-mm LB/agarose/kanamycin plates. The colonies will be scraped off plates and amplified in 100 ml of LB/kan broth. The recovered plasmid will be subjected to 2-5 rounds of selection and individual colonies will be picked for further evaluation.

DEAE Dextran Transfection Method

Cells will be plated at 3×10⁵ cells/well on 6-well plates and transfected 24 hours later. 1-2 μg of DNA/well will be used for HeLa and 0.25-0.5 μg of DNA/well for COS-1. DNA will be added to 490 μL of PBS (Ca⁺⁺, Mg⁺⁺-free) containing 250 μg/ml DEAE-dextran. Wells will be washed twice with 2 mls of PBS (Ca⁺⁺, Mg⁺⁺-free). 500 μL of the DNA/DEAE-dextran/PBS mixture will be added and then incubated for 30 minutes at 37° C., with rocking every 5 minutes. 2 mLs of media (DMEM w/ 10% FBS, 1% P/S) containing 80 μM Chloroquine will be added and then incubated an additional 2.5 hours at 37° C. The media will be removed and the cells shocked with 1 mL of 10% DMSO in media (DMEM w/10% FBS, 1% P/S) for 1 hour. The DMSO solution will be removed and replaced with 2 mLs of media (DMEM w/10% FBS, 1% P/S). If not treating cells, the assay will be performed 48 hours after transfection for HeLa and 24 hours after for COS-1 cells.

Luciferase Assay

Transfected cells will be harvested from plates using trypsin/EDTA and detergent extract using described conditions (Promega). Excised tumors will be homogenized in the same extraction buffer by bead homogenization (Biospec, Bartlesville, Okla.) Twenty microliters of extract will be added in duplicate to individual wells of an opaque 96-well plate (Microlite-1, Dynex Technologies, Inc., Chatilly, Va.). Relative light units (RLU) of wells will be determined by 30 second readings on a 96-well luminometer (Berthold Microlumat, LB96P, Wallac Inc., Gaithersburg, Md.).

Immunoassay of Chloramphenical Acetyl Transferase (CAT)

In vitro transfected cells will be harvested from plates using trypsin/EDTA solution and lysed with detergent buffers supplied with the CAT immunoassay kit (Boehringer Mannhein, Indianapolis, Ind.). Tumor extracts will be prepared by placing tissue in a 2.0 mL screw-cap polypropylene tubes containing 0.25 gram of zirconium beads (Biospec, Bartlesville, Okla.) and snap frozen in liquid nitrogen for storage at −80EC. Protein will be extracted from tissue by bead homogenization (Biospec) with 1.0 mL ice-cold extraction buffer (50 mM Tris pH 8.0, 150 mM NaCl, 1 mM EDTA, 0.5% Triton X-100, 1 mM pepstatin A, 0.25 mM PMSF and 10 mM leupeptin). The tubes will be centrifuged for 10 minutes at 4EC prior to immunoassay. The sensitivity of the assay is ˜10 pg/ml, and the range is 15.6-1000 pg/ml.

RNA extraction and Northern Blot Analysis

Total RNA from cells and tissue will be extracted using RNAzo™ (Tel-Test Inc., Friendswood, Tex.). The RNA pellet obtained after isopropanol precipitation will be washed with 70% ethanol and resuspended in 50 μl of DEPC-treated water. DNA will be removed from the RNA by incubating with DNase (Ambion, Austin, Tex.) at 37° C. for 30 minutes. The RNA will be precipitated with ethanol, washed, and resuspended in RNase-free water. The yield will be determined spectrophotometrically at 260/280 nm. Total mRNA will be denatured with glyoxal and electrophoresed in a 1.2% agarose/MOPS gel system for 4 hours. RNAs will be transferred to nylon membranes and probed with ³²P-labelled specific luciferase, GFP or GAPDH plasmid fragments. Blots will be washed first with low stringency, followed by high stringency washes. Probe specific radioactivity will be quantitated on a phosphoimager.

DNA Extraction and Southern Blot Analysis

Total DNA will be isolated from cells and tissue using conventional molecular biology techniques. DNA will be digested with restriction endonucleases, electrophoresed in 0.8% agarose/TBE gels, and transferred to nylon membranes. Blots will be probed with ³²P-labelled specific luciferase, GFP or GAPDH plasmid fragments. Blots will be washed with low stringency, followed by high stringency washes. Probe specific radioactivity will be quantitated on a phosphoimager.

Cell Lines

Renca, SCCVII and A549 cell lines will be obtained from the ATCC and maintained in culture according to the suppliers directions. The SCCVII cell line is squamous cell carcinoma that spontaneously arose in the C3H/HeN strain (Suit et al., Radiat. Res., 104:47-65, 1985). This cell line is passaged by short-term culture prior to in vivo tumor implants. The Renca cell line is spontaneously derived renal cell carcinoma derived from Balb/c mice (Salup et al., J. Immunol. 138:641-647, 1987). The A549 cell line is derived from human lung tumor epithelium (Lieber et al., Int. J. Cancer, 17(1):62-70, 1976.

Animal Models

Animals will be housed in microisolators in the Laboratory Animal Resource (LAR) vivarium at GENEMEDICINE, INC. and maintained on ad lib rat chow and water (temperature 23EC, humidity 40%, light-dark cycle 12hrs-12hrs). Animals will arrive from vendor (Charles River) and be acclimated for a period of at least 7 days prior to initiation of the study. Renca tumor cells (7.5×10⁵) will be implanted subcutaneously in 30 μL in syngeneic Balb/c mice and allowed to grow to a 20-30 mm³ size. SCCVII tumors cells (4×10⁵) will be implanted subcutaneously in 30 μL in syngeneic C3H mice and allowed to grow to a 20-30 mm³ size. Tumors will be injected with 50 μL of plasmid formulations. Tumors will be excised at various time points following injection.

### Example 3

Construction of Novel Promoters with an Enhanced Duration of Transgene Expression in Tumor Cells in vitro

Isolate Novel Plasmids Using a Cell-Based Selection System

A general technique for screening a combinatorial library of plasmids with improved performance has been designed. Random mutagenesis of a plasmid region of interest coupled with a high throughput screening protocol for detection of cell surface proteins on transfected mammalian cells is a powerful means to detect novel sequences in a highly complex mixture of plasmids (Seed and Aruffo, Proc. Natl. Acad. Sci, USA, 84:3365, 1987; Aruffo and Seed, Proc. Natl. Acad. Sci. 84:8573, 1987 and Aruffo and EMBO J., 6:3313, 1987). One advantage over previously established methods is the use of reporter genes detectable without radiolabelled or fluorochrome-labelled ligands or antibodies. Green fluorescent protein gene, isolated from the jellyfish Aequorea victoria, encodes a protein that fluoresces upon excitation with blue-green light (Chalfie, Photochem Photobiol, 62:651, 1995; Chalfie et al., Science, 263:802, 1994). This gene is a useful reporter or marker since the protein when expressed intracellularly spontaneously fluoresces without added cofactors.

Fluorescence can be detected by flow cytometry in mammalian cells transfected with vectors containing the GFP transgene. The GFP cDNA has been further optimized for human codon usage (Zolotukhin et al., J. Virol., 70:4646, 1996), and a S65T mutation was introduced to improve the fluorescence signal (Heim and Tsien, Curren. Biol. 6:178, 1996). Cells expressing GFP have been sorted and viably recovered using flow cytometry (Dell'Arciprete et al., J. Histochem. Cytochem. 44:629, 1996). Thus, sorting and recovering plasmids expressing GFP is a powerful method to screening random mutations that improve the transgene expression level.

The general approach is: (1) transfect cells with a combinatorial promoter library ligated into a green fluorescent protein (GFP) expression vector, (2) culture transfected cells in the presence of cytokines, (3) select GFP expressing cells by FACS sorting, (4) isolate episomal DNA fraction from cells sorted for high GFP expression.

This method can be used to select expression plasmids following in vitro or preferably from in vivo transfected cells that are exposed to a more relevant cytokine-rich environment. An example of cell-based plasmid selection involves a GFP expression plasmid that can be isolated from cells transfected with a 1:10,000 (GFP:non-GFP) dilution of control plasmid.

Combinatorial libraries have been constructed which consist of the pGF1187 plasmid containing a random library of oligonucleotides derived from the DNA sequence of the human CMV immediate early enhancer or specific transcription factor binding sites. From the libraries constructed using the established cell-based selection system, isolate plasmids that express transgene in the presence of cytokines. Both human and mouse cell lines will be used to screen the libraries. Candidate plasmids will be isolated and further characterized by Northern blot analysis of transfected cells and reporter assays.

### Example 4

Demonstration of Transfection Facilitating Agent-Plasmid DNA Complex Formation: Preparation of PVP Formulated Nucleic Acid Molecules

Concentrated pDNA stock solutions were made by lyophilizing and rehydrating pDNA with water to a final pDNA concentration of 3-5 mg/ml. Formulations were made by aliquoting appropriate volumes of sterile stock solutions of pDNA, 5M NaCL, and polymer to obtain a final pDNA concentration in an isotonic polymer solution. Stock solutions were added in the following order: water, plasmid, polymer, and 5M NaCl. The plasmid and polymers were allowed to incubate at room temperature for 15 minutes prior to adding salt or lactose for ionicity adjustments. Likewise, Na-citrate buffers in 0.9% NaCl were added after incubating the plasmid and polymers for 15 minutes at room temperature. The osmotic pressure of selected formulations was measured (n=3) using a Fiske One-Ten Micro-Sample Osmometer. The pH of all formulations was measured using an Accumet Model 15 pH Meter and the viscosity of all formulations was measured using a Programmable Rheometer Model DV-III.

Dynamic dialysis was used with various interactive polymer formulations to measure binding between PVP and plasmid DNA. One ml of formulations and corresponding controls were place in prewashed dialysis sacs. The dialysis sacs were closed and suspended in stirred saline solutions (100 ml) at 25° C. One ml aliquots were taken from the acceptor compartment over time and replaced with fresh media. The concentration of PVP in the diffused samples collected over time was measured spectroscopically at 220 nm.

In all cases, the rate of PVP diffusion through the dialysis membrane was decreased in the presence of plasmid DNA, indicating complex formation between PVP and plasmid DNA. The reduction in the diffusion rate for PVP in the presence of plasmid DNA was directly proportional to the initial amount of PVP in the dialysis sac. It was also determined that the sac volume remained constant during the duration of the experiment and that adherence of PVP to the membrane was negligible.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

What is claimed is:

1. A modified cytomegalovirus promoter that enhances the duration or intensity of expression of a desired gene in the presence of one or more cytokines relative to the expression produced by the corresponding non-modified cytomegalovirus promoter in the presence of said one or more cytokines.

2. The modified cytomegalovirus promoter of claim 1, wherein said modified cytomegalovirus promoter comprises a nucleic acid sequence that is substantially similar to the nucleic acid sequence encoding said non-modified cytomegalovirus promoter.

3. The modified cytomegalovirus promoter of claim 1, wherein said modified cytomegalovirus promoter lacks one or more response elements present in said non-modified cytomegalovirus promoter and which interferes with maximal expression of said desired gene when in the presence of said one or more cytokines.

4. The modified cytomegalovirus promoter of claim 1, wherein said modified cytomegalovirus promoter lacks a number of contiguous nucleic acids from the 3′ or 5′ end of said non-modified cytomegalovirus promoter.

5. The modified cytomegalovirus promoter of claim 1, wherein said one or more cytokines are inflammatory cytokines.

6. The modified cytomegalovirus promoter of claim 1, wherein said modified cytomegalovirus promoter enhances expression of said desired gene in vitro.

7. The modified cytomegalovirus promoter of claim 1, wherein said modified cytomegalovirus promoter enhances expression of said desired gene in vivo.

8. The modified cytomegalovirus promoter of claim 1, wherein said modified cytomegalovirus promoter enhances the duration of expression of said desired gene.

9. The modified cytomegalovirus promoter of claim 8, wherein said duration of expression is increased at least two-fold.

10. The modified cytomegalovirus promoter of claim 1, wherein said modified cytomegalovirus promoter enhances the amount of expression of said desired gene.

11. The modified cytomegalovirus promoter of claim 1, wherein said modified cytomegalovirus promoter has the nucleic acid sequence of the modified cytomegalovirus promoter of plasmid pLC1001 or pLC0888.

12. The modified cytomegalovirus promoter of claim 1, wherein said modified cytomegalovirus promoter is a modified human cytomegalovirus promoter.

13. The modified cytomegalovirus promoter of claim 1, provided that said modification is not a mutation in a binding site for a Gfi-1 transcription repressor.

14. The modified cytomegalovirus promoter of claim 1, wherein said desired gene encodes a compound selected from the group consisting of IL-12, interferon alpha, and TNF alpha.

15. The modified cytomegalovirus promoter of claim 1, made by a process comprising the step of specifically deleting sequences from the non-modified cytomegalovirus promoter which interfere with maximal expression of said desired gene when in the presence of said one or more cytokines.

16. The modified cytomegalovirus promoter of claim 1 made by a process comprising the steps of: (a) transfecting cells with a combinatorial library of modified cytomegalovirus promoters ligated into a green fluorescent protein expression vector, wherein said combinatorial library comprises a series of modified cytomegalovirus promoters, wherein each modified cytomegalovirus promoter in said series comprises an independently modified form of a corresponding non-modified cytomegalovirus promoter; (b) culturing the transfected cells in the presence of one or more cytokines; (c) selecting GFP expressing cells by FACS sorting; and (d) isolating episomal DNA fraction from cells sorted for high GFP expression.

17. The promoter of claim 1, wherein said selection is performed in vivo.

18. A method of making a modified cytomegalovirus promoter of any one of claims 1-14, comprising the step of specifically deleting sequences from the non-modified cytomegalovirus promoter which interfere with maximal expression of said desired gene when in the presence of said one or more cytokines.

19. A method of making a modified cytomegalovirus promoter of any one of claims 1-14, comprising the steps of: (a) transfecting cells with a combinatorial library of modified cytomegalovirus promoters ligated into a green fluorescent protein expression vector, wherein said combinatorial library comprises a series of modified cytomegalovirus promoters, wherein each modified cytomegalovirus promoter in said series comprises an independently modified form of a corresponding non-modified cytomegalovirus promoter; (b) culturing the transfected cells in the presence of one or more cytokines; (c) selecting GFP expressing cells by FACS sorting; and (d) isolating episomal DNA fraction from cells sorted for high GFP expression.

20. The method of claim 18, wherein said transfection restored in vivo.

21. A method of making a modified cytomegalovirus promoter of any one of claims 1-14, comprising the step of chemically synthesizing said modified cytomegalovirus promoter.

22. A method of using a modified cytomegalovirus promoter of any one of claims 1-17, comprising the step of expressing said desired gene in the presence of said one or more cytokines.

23. In a method of expressing a desired gene in the presence of one or more cytokines, the improvement comprising using a modified cytomegalovirus promoter of any one of claims 1-17.

24. A vector or plasmid comprising a modified cytomegalovirus promoter of any one of claims 1-17 and said desired gene.

25. A method of making a vector or plasmid comprising a modified cytomegalovirus promoter of any one of claims 1-17 and said desired gene comprising the step of combining said modified cytomegalovirus and said desired gene.

26. A method of using a vector or plasmid comprising a modified cytomegalovirus promoter of any one of claims 1-17 and said desired gene comprising the step of expressing said desired gene in the presence of said one or more cytokines.

27. A combinatorial library, of modified cytomegalovirus promoters comprising a series of modified cytomegalovirus promoters, wherein each modified cytomegalovirus promoter in said series comprises an independently modified form of a corresponding non-modified cytomegalovirus promoter.

28. A method of making a combinatorial library of claim 22, comprising the step of making a series of modified cytomegalovirus promoters, wherein each modified cytomegalovirus promoter in said series comprises an independently modified form of a corresponding non-modified cytomegalovirus promoter.

29. A method of using a combinatorial library of claim 27, comprising the step of exposing each member of said series to one or more test agents.

30. A method of screening modified cytomegalovirus promoters for those that enhance the duration or intensity of expression of a desired gene in the presence of one or more cytokines relative to the expression produced by the corresponding non-modified cytomegalovirus promoter in the presence of said one or more cytokines comprising the step of using said modified cytomegalovirus promoter to express said desired gene in the presence of said one or more cytokines and comparing the expression to that produced using said non-modified promoter in the presence of said one or more cytokines.

31. The method of claim 28, wherein said method comprises the step of using a combinatorial library of modified cytomegalovirus promoters to express said desired gene in the presence of said one or more cytokines and comparing the expression to that produced using said non-modified promoter in the presence of said one or more cytokines, wherein said combinatorial library comprises a series of modified cytomegalovirus promoters, wherein each modified cytomegalovirus promoter in said series comprises an independently modified form of a corresponding non-modified cytomegalovirus promoter.

32. The method of claim 28, wherein said screening comprises FACS/MACS cell selection.

33. A method of treating or preventing a disease using a vector or plasmid comprising a modified cytomegalovirus promoter of any one of claims 1-15 and said desired gene.

34. A formulation comprising a vector or plasmid of claim 24 and a transfection facilitating agent.

35. The formulation of claim 34, wherein said transfection facilitating agent is a protective/interactive non-condensing compound.

36. A method for delivering the formulation of claim 34, wherein said delivery is to the cells of a mammal.

37. The method of claim 36, wherein said delivery is performed by a needle-free injection device or by a pulse-voltage delivery device.

38. A method of making a formulation of claim 34 comprising the step of combining said vector or plasmid and said transfection facilitating agent.


SEQUENCE LISTING (30 sequences)

SEQ ID NO: 1 - DNA, 242 bp; single-stranded, linear
gtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 2 - DNA, 560 bp; single-stranded, linear
aattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcc

SEQ ID NO: 3 - DNA, 27 bp; single-stranded, linear
cgatgagcttgcatgcctgtattagat

SEQ ID NO: 4 - DNA, 27 bp; single-stranded, linear
cgatctaatacaggcatgcaagctcat

SEQ ID NO: 5 - DNA, 27 bp; single-stranded, linear
cgatgtcatcgctattaccatcatgat

SEQ ID NO: 6 - DNA, 26 bp; single-stranded, linear
cgtcatgatggtaatagcgatgacat

SEQ ID NO: 7 - DNA, 27 bp; single-stranded, linear
cgatgtgatgcggttttggcagtacat

SEQ ID NO: 8 - DNA, 26 bp; single-stranded, linear
cgtgtactgccaaaaccgcatcacat

SEQ ID NO: 9 - DNA, 25 bp; single-stranded, linear
cgatcatcaatgggcgtggatgcat

SEQ ID NO: 10 - DNA, 24 bp; single-stranded, linear
cgtgcatccacgcccattgatgat

SEQ ID NO: 11 - DNA, 26 bp; single-stranded, linear
cgatgtttgactcacggggatttcat

SEQ ID NO: 12 - DNA, 25 bp; single-stranded, linear
cgtgaaatccccgtgagtcaaacat

SEQ ID NO: 13 - DNA, 26 bp; single-stranded, linear
cgatcaagtctccaccccattgacat

SEQ ID NO: 14 - DNA, 25 bp; single-stranded, linear
cgtgtcaatggggtggagacttgat

SEQ ID NO: 15 - DNA, 30 bp; single-stranded, linear
cgatgtcaatgggagtttgttttggcacat

SEQ ID NO: 16 - DNA, 29 bp; single-stranded, linear
cgtgtgccaaaacaaactcccattgacat

SEQ ID NO: 17 - DNA, 25 bp; single-stranded, linear
cgatcaaaatcaacgggactttcat

SEQ ID NO: 18 - DNA, 24 bp; single-stranded, linear
cgtgaaagtcccgttgattttgat

SEQ ID NO: 19 - DNA, 25 bp; single-stranded, linear
cgatcaaaatgtcgtaacaactcat

SEQ ID NO: 20 - DNA, 24 bp; single-stranded, linear
cgtgagttgttacgacattttgat

SEQ ID NO: 21 - DNA, 23 bp; single-stranded, linear
cggccaggcgggccatttaccgt

SEQ ID NO: 22 - DNA, 23 bp; single-stranded, linear
cgacggtaaatggcccgcctggc

SEQ ID NO: 23 - DNA, 21 bp; single-stranded, linear
cgccccattgacgtcaatggg

SEQ ID NO: 24 - DNA, 21 bp; single-stranded, linear
cgcccattgacgtcaatccgg

SEQ ID NO: 25 - DNA, 20 bp; single-stranded, linear
cgactaacgggactttccaa

SEQ ID NO: 26 - DNA, 20 bp; single-stranded, linear
cgttggaaagtcccgttagt

SEQ ID NO: 27 - DNA, 20 bp; single-stranded, linear
cgcgcttgacagtacatcaa

SEQ ID NO: 28 - DNA, 20 bp; single-stranded, linear
cgttgatgtactgtcaagcg

SEQ ID NO: 29 - DNA, 18 bp; single-stranded, linear
cgnnnnnccgcccnnnnn

SEQ ID NO: 30 - DNA, 18 bp; single-stranded, linear
cgnnnnngggcggnnnnn